(19) United States
(12) Patent Application Publication
(10) Pub. No.: US 2026/0317364 A1
(43) Pub. Date: Oct. 15, 2026

(54) ACTIVE MACROLIDE ANTIBIOTIC COMPOUNDS AGAINST PATHOGENS

(71) Applicants: YEDA RESEARCH AND DEVELOPMENT CO. LTD., Rehovot (IL); RUHR-UNIVERSITÄT BOCHUM, Bochum (DE)

(72) Inventors: Ada YONATH, Rehovot (IL); Anat BASHAN, Rehovot (IL); Ella ZIMMERMAN, Rehovot (IL); Disha Gajanan HIREGANGE, Rehovot (IL); Andre RIVALTA; Frank SCHULZ, Hattingen (DE); Sascha HEINRICH, Bochum (DE); David MÖLLER, Witten (DE); Niclas PRYK, Dortmund (DE)

(73) Assignees: YEDA RESEARCH AND DEVELOPMENT CO. LTD., Rehovot (IL); RUHR-UNIVERSITÄT BOCHUM, Bochum (DE)

(21) Appl. No.: 19/484,973

(22) PCT Filed: May 23, 2024
(86) PCT No.: PCT/IL2024/050506

§ 371 Date: Nov. 17, 2025

(30) Foreign Application Priority Data
May 23, 2023 (IL) 303173

(51) Int. Cl. C07H 17/00; A61K 31/7048; A61K 31/7056; A61P 31/04; C07H 17/08

(52) U.S. Cl. CPC C07H 17/00; A61K 31/7048; A61K 31/7056; A61P 31/04; C07H 17/08

(57) ABSTRACT

The present invention relates to macrolide antibiotic compounds and use thereof for treatment of infections due to S. aureus resistant strains or E. coli strains.

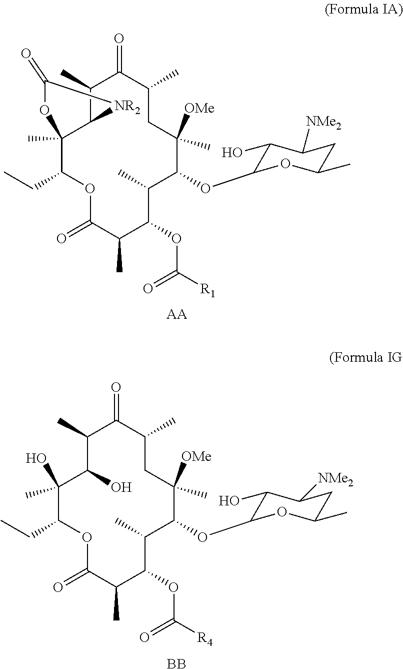

### FIELD OF THE INVENTION

The present invention relates to macrolide antibiotic compounds and use thereof for treatment of infections due to S. aureus resistant strains or E. coli strains.

### BACKGROUND OF THE INVENTION

Macrolide antibiotics such as erythromycin or clarithromycin have been among the most important drugs for the treatment of community-acquired respiratory tract infections. The increasing number of multi-resistant pathogens hinders their application in the treatment of infections.

A variety of synthetic strategies have been used to identify candidate compounds which overcome these resistance mechanisms. Recent results on the mode of action of ketolides revealed that the efficiency of ketolides to kill bacteria instead of solely inhibiting the growth is caused by increased time of ribosomal interaction not due to enhanced binding affinity. One alternative to ketolides, which are often hampered by side-effects, is the development of acylide antibiotics in recent years. There is an ongoing need for novel hybrid antibiotics composed of acylide and ketolide moieties that would show a remarkable increase in antibacterial activity against inducible macrolide-resistant strains.

### SUMMARY OF THE INVENTION

In one aspect, the present invention provides a compound represented by the structure of Formula IA:

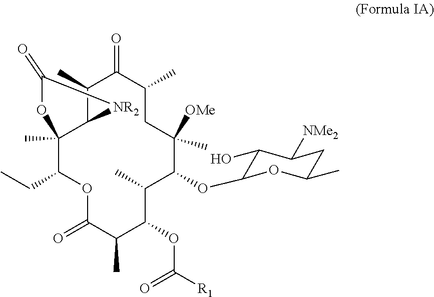

wherein
R₁ is

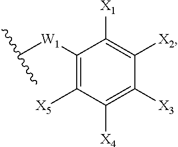

alkyl, alkene, aryl, heteroaryl, -alkylene-N₃, -alkylene-cycloalkyl, alkylene-heterocycloalkyl-aryl, alkylene-heterocycloalkyl, or -alkylene-heteroaryl;
X₁-X₅ are each independently selected from H, halo, CN, NO₂, OH, alkoxy —O-alkylene-aryl, —O-alkylene-heteroaryl, —N-alkylene-aryl, —N-alkylene-heteroaryl, —SO₂-alkylene-aryl, and —SO₂-alkylene-heteroaryl; or two of X₁-X₅ are connected via —O—(CH₂)ₘ—O—;
m is an integer from 1-10;
W₁ is a bond, alkylene, alkenylene, alkynylene, cycloalkyl, heterocyclic, aryl, heteroaryl, or an ether group;
R₂ is H or

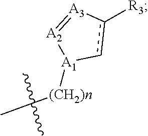

[symbol] represents a single or double bond;
n is an integer from 0-10;
R₃ is heteroaryl or aryl;
A₁, A₂, and A₃ are each independently N, NH, NR′, C, CH, CR″, CHR″, CR″R′″ or CH₂; and
R′, R″, and R′″ are each independently alkyl, cycloalkyl, aryl, heteroaryl, NO₂, CN, hydroxyl or halo.

In one aspect, the present invention provides a compound represented by the structure of Formula IG:

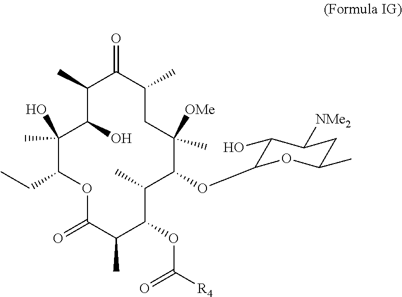

wherein
R₄ is

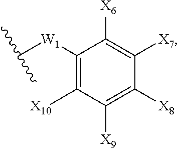

alkyl, alkene, aryl, heteroaryl, alkylene-N₃, alkylene-cycloalkyl, alkylene-heterocycloalkyl-aryl, alkylene-heterocycloalkyl, alkylene-heteroaryl, -alkylene-heteroaryl-aryl, -alkylene-heteroaryl-alkylene-heterocycloalkyl, -alkylene-heteroaryl-alkylene-aryl, -alkylene-aryl-alkylene-aryl, or -alkylene-heteroaryl-alkylene-heteroaryl;
X₆-X₁₀ are each independently selected from H, halo, CN, OH, NO₂, O-alkylene aryl, —O-alkylene-heteroaryl, —N-alkylene-aryl, —N-alkylene-heteroaryl, —SO₂-alkylene-aryl, and —SO₂— alkylene-heteroaryl; or two of X₆-X₁₀ are connected via —O—(CH₂)ₘ—O—;
m is an integer from 1-10; and
W₁ is a bond, alkylene, alkenylene, alkynylene, cycloalkyl, heterocyclic, aryl, heteroaryl, or an ether group.

In another aspect, the present invention provides a pharmaceutical composition comprising a compound of the invention as described herein and a pharmaceutically acceptable carrier.

In some embodiments, the pharmaceutical composition of the invention is for use in inhibiting selectively S. aureus resistant strains, wherein the strain is DSM20231, BAA976, or BAA977.

In some embodiments, the pharmaceutical composition of the invention is for use in inhibiting selectively of E. coli strain, wherein the strain is tolC; MB5747.

In some embodiments, the pharmaceutical composition of the invention is for use in the treatment of an infection, wherein the infection is due to S. aureus resistant strains, and wherein the strain is DSM20231, BAA976, or BAA977.

In other embodiments, the pharmaceutical composition of the invention is for use in the treatment of an infection, wherein the infection is due to E. coli strain, and wherein the strain is tolC; MB5747.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts MIC values of compounds 2-24 compared to macrolide antibiotics clarithromycin (CTY), telithromycin (TEL), solithromycin (SOL), acylide TEA0777, and acylide X10324 against four Gram-positive and nine Gram-negative bacterial as well as two yeast strains and two human cell types.

FIG. 2 depicts MIC values of compounds 29-42 compared to macrolide antibiotics clarithromycin (CTY), telithromycin (TEL), solithromycin (SOL), TEA0777, and X10324 against four Gram-positive and nine Gram-negative bacterial as well as two yeast strains and two human cells.

FIG. 3 depicts MICs of compounds 47,48 (SOL-based), 55, and 56 (TEL-based) compared to macrolide antibiotics against bacterial strains. The majority of the tested acylides showed potent antibacterial activity against S. aureus except for the constitutively resistant MRSA-strain ATCC 43300. Compound 50 is 2-desfluoro-solithromycin.

FIG. 4 provides the chemical structures of acylide compounds X10324, 40, 48, and 56.

FIGS. 5A-5F depict the location within the SA50S of the four compounds under study, X10324, 40, 48, 56. (FIG. 5A) SA50S in ribbon representation in complex with the four overlapped compounds viewed as spheres. (FIG. 5B) Superposition of X10324, 40, 48, and 56 structures from each complex shows high similarity in the overall orientation of the molecules. (FIGS. 5C-5F) Superimposing of X10324 (FIG. 5C), 40 (FIG. 5D), 48 (FIG. 5E) and 56 (FIG. 5F) on their respective EM density maps. The maps contour level is 6=2.

FIGS. 6A-6D depict multiple possible orientations for the 56 acylide: main orientation (FIG. 6A), alternate orientation of the alkyl-aryl side chain (FIG. 6B), alternate orientation of the F-ring-F moiety (FIG. 6C), alternate orientations of both the alkyl-aryl side chain and the 2,6-difluoro-4-nitro phenylacetic acid moiety (FIG. 6D).

FIG. 7 depicts the superposition of X10324 on the ery macrolide from different PDB structures (6ND6 from T. thermophilus, 7NSO from E. coli, and 4WFN from S. aureus). The typical macrolide lactone ring as well as the desosamine sugar are clearly overlapping whereas the cladinose is substituted by the 2,6-difluoro-4-nitro phenylacetic acid moiety.

FIG. 8 depicts superposition of 48 with sol (from PDB 4WWW). The two compounds share the same alkyl-aryl side chain, whereas the typical ketone group of sol and other ketolides is substituted by the 2,6-difluoro-4-nitro phenylacetic acid moiety.

FIG. 9 depicts the superposition of 56 in two orientations (main; alternate) with TEL from different PDB structures (6XHY from T. thermophilus, 7NSQ from E. coli, 4WF9 from S. aureus, 1YIJ from H. marismortui). The alkyl-aryl side chain, identical in the two compounds, appears to be flexible. The 2,6-difluoro-4-nitro phenylacetic acid moiety shows also flexibility.

FIGS. 10A-10D depict a view of the binding pocket of X10324 (FIG. 10A), 40 (FIG. 10B), 48 (FIG. 10C), and 56 (FIG. 10D) at the ribosome NPET (thick grey). Hydrogen bonds are marked with black dashes; all compounds share the same H-bond with a water molecule (circle/ball) and nucleotide A2058, similarly to other macrolides. In addition, all compounds appear to have a stacking interaction between the 2,6-difluoro-4-nitro phenylacetic group and nucleotide G2505.

FIGS. 11A-11D depict the superposition of the apo SA50S structure (PDB code 6HMA) and the SA50S-X10324 (FIG. 11A), SA50S-40 (FIG. 11B), SA50S-48 (FIG. 11C), and SA50S-56 (FIG. 11D). The movement of nucleotides G2505 and U2506 from the native to the compound-bound state is shown with a black arrow.

FIG. 12 depicts a summary of the MIC values [mg/ml] of the growth inhibition assay using gram negative and gram-positive strains.

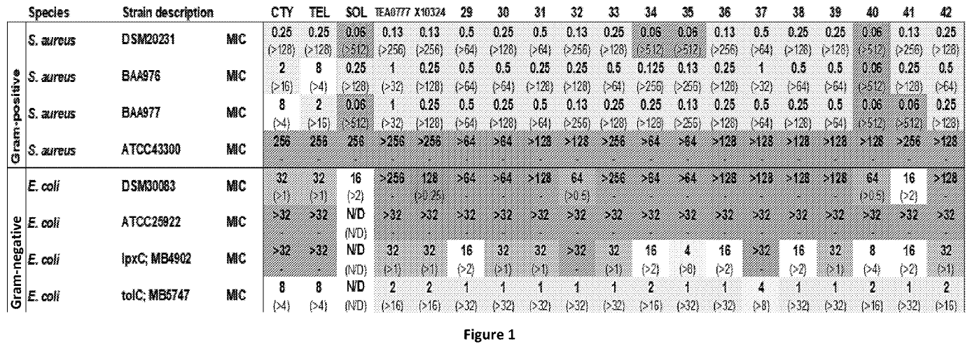

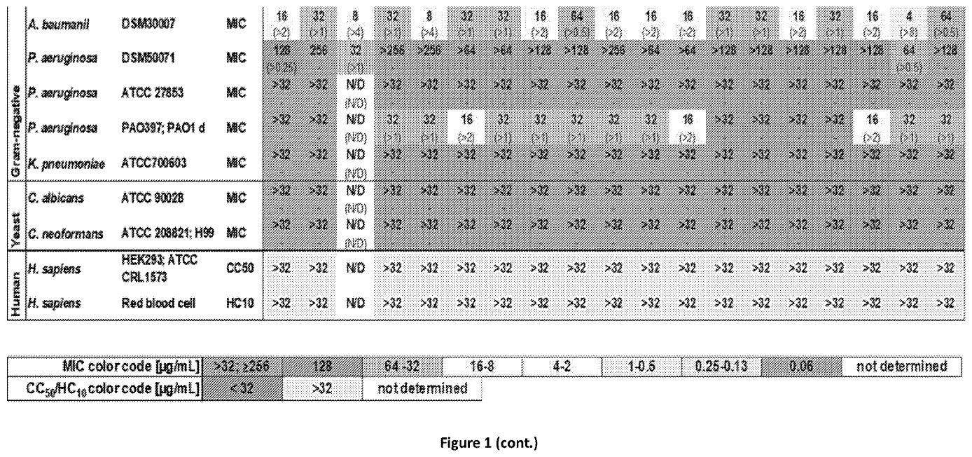

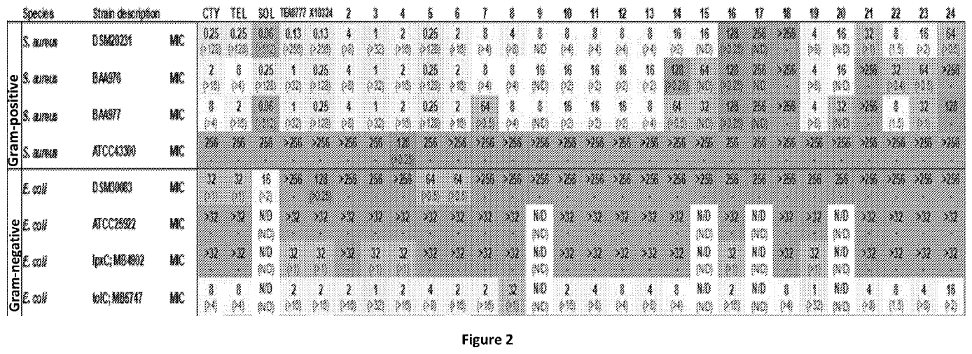

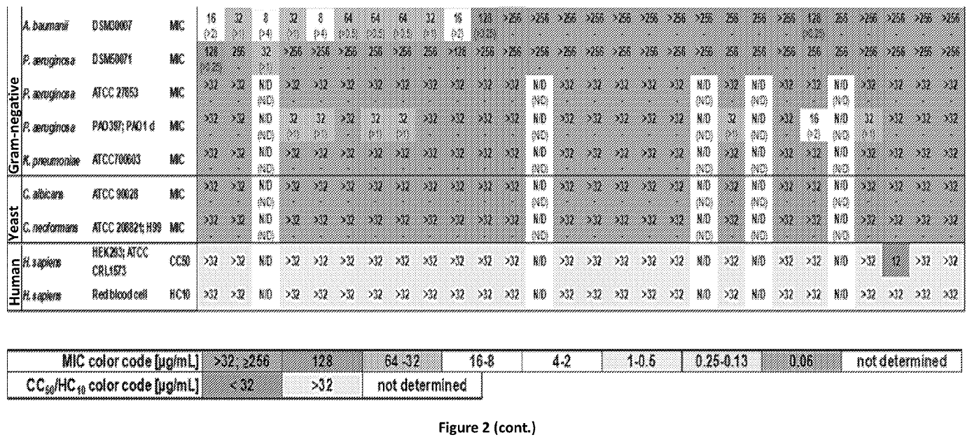

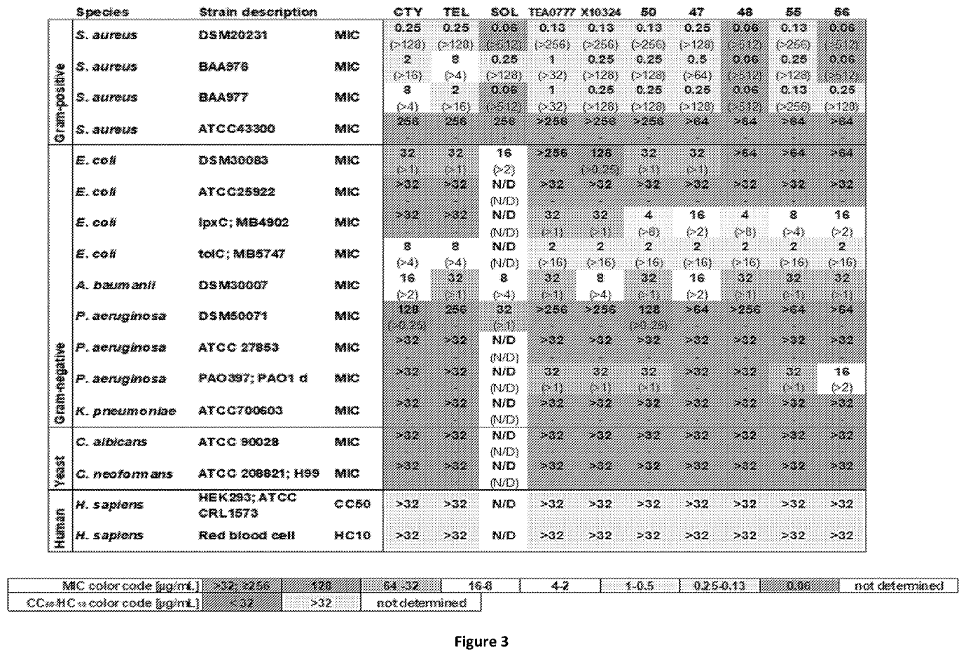

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one aspect, the present invention provides a compound represented by the structure of Formula IA:

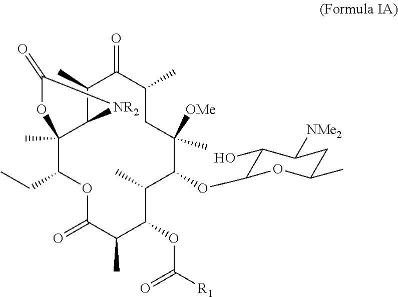

wherein
R₁ is

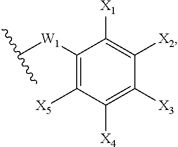

alkyl, alkene, aryl, heteroaryl, alkylene-N₃, -alkylene-cycloalkyl, -alkylene-heterocycloalkyl-aryl, -alkylene-heterocycloalkyl, or -alkylene-heteroaryl;
X₁-X₅ are each independently selected from H, halo, CN, NO₂, OH, alkoxy, —O-alkylene-aryl, —O-alkylene-heteroaryl, —N-alkylene-aryl, —N-alkylene-heteroaryl, —SO₂-alkylene-aryl, and —SO₂— alkylene-heteroaryl; or two of X₁-X₅ are connected via —O—(CH₂)ₘ—O—;
m is an integer from 1-10;
W₁ is a bond, alkylene, alkenylene, alkynylene, cycloalkyl, heterocyclic, aryl, heteroaryl, or an ether group;
R₂ is H or

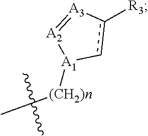

[symbol] represents a single or double bond;
n is an integer from 0-10;
R₃ is heteroaryl or aryl;
A₁, A₂, and A₃ are each independently N, NH, NR′, C, CH, CR″, CHR″, CR″R′″ or CH₂; and
R′, R″, and R′″ are each independently alkyl, cycloalkyl, aryl, heteroaryl, NO₂, CN, hydroxyl or halo.

In other aspects, the present invention provides a compound represented by the structure of Formula IA, wherein if R₂ is H, R₁ is not —CH₂-pyridine. In other aspects, at least one of X₁-X₅ is not H. In other aspects if R₂ is H, then R₁ is not p —CH₂—C₆H₄—NO₂ (para-nitrobenzyl). In other aspects R₃ is not pyrimidine.

In other aspects, the present invention provides a compound represented by the structure of Formula IA,

wherein
R₁ is

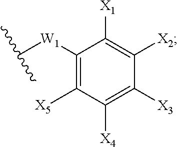

X₁-X₅ are each independently selected from H, halo, CN, NO₂, OH, —O-alkylene-aryl, —O-alkylene-heteroaryl, —N-alkylene-aryl, —N-alkylene-heteroaryl, —SO₂-alkylene-aryl, and —SO₂-alkylene-heteroaryl; or two of X₁-X₅ are connected via —O—(CH₂)ₘ—O—; wherein at least one of X₁-X₅ is not H; wherein if R₂ is H, R₁ is not p —CH₂—C₆H₄—NO₂;
m is an integer from 1-10; and
W₁ is an alkylene.
R₂ is H or

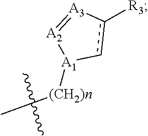

[symbol] represents a single or double bond;
n is an integer from 0-10;
R₃ is heteroaryl or aryl, wherein R₃ is not pyrimidine;
A₁, A₂, and A₃ are each independently N, NH, NR′, C, CH, CR″, CHR″, CR″R′″ or CH₂; and R′, R″, and R′″ are each independently alkyl, cycloalkyl, aryl, heteroaryl, NO₂, CN, hydroxyl, or halo.

In one aspect, the present invention provides a compound represented by the structure of Formula IB:

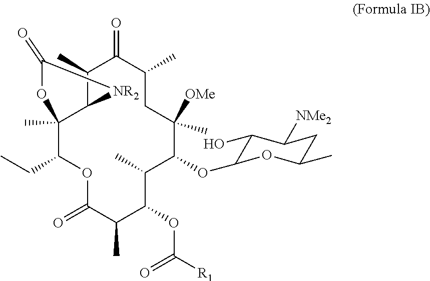

wherein R₁ is as defined in the structure of Formula IA.

In one aspect, the present invention provides a compound represented by the structure of Formula IC:

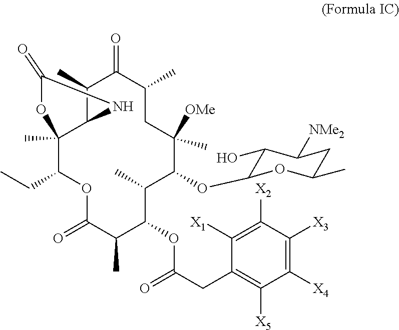

wherein X₁-X₅ are as defined in the structure of Formula IA.

In some embodiments, the compound of Formula IB is represented by the structure of Compounds 29-42:

TABLE 1

| Compound's | |
|---|---|
| Number | R₁ of Formula IB |
| 29 | 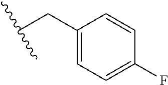 [SMILES: *Cc1ccc(F)cc1] |
| 30 | 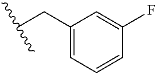 [SMILES: *Cc1cccc(F)c1] |
| 31 | 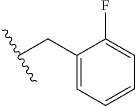 [SMILES: *Cc1ccccc1F] |
| 32 | 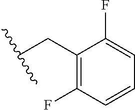 [SMILES: *Cc1c(F)cccc1F] |
| 33 | 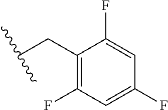 [SMILES: *Cc1c(F)cc(F)cc1F] |
| 34 | 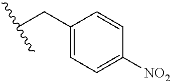 [SMILES: *Cc1ccc([N+](=O)[O-])cc1] |
| 35 | 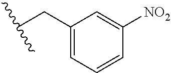 [SMILES: *Cc1cccc([N+](=O)[O-])c1] |
| 36 | 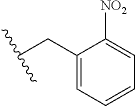 [SMILES: *Cc1ccccc1[N+](=O)[O-]] |
| 37 | 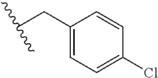 [SMILES: *Cc1ccc(Cl)cc1] |
| 38 | 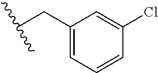 [SMILES: *Cc1cccc(Cl)c1] |
| 39 | 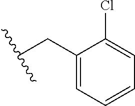 [SMILES: *Cc1ccccc1Cl] |
| 40 | 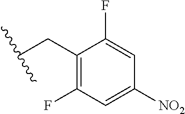 [SMILES: *Cc1c(F)cc([N+](=O)[O-])cc1F] |
| 41 | 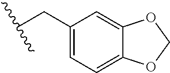 [SMILES: *Cc1ccc2c(c1)OCO2] |
| 42 | 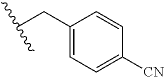 [SMILES: *Cc1ccc(C#N)cc1] |

In one aspect, the present invention provides a compound represented by the structure of Formula ID:

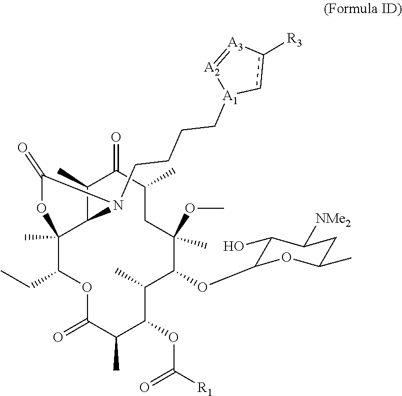

wherein R₁, R₃, A₁-A₃ and [symbol] are as defined in the structure of Formula IA.

In one aspect, the present invention provides a compound represented by the structure of Formula IE:

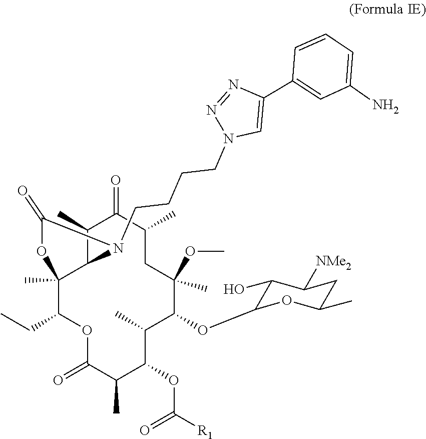

wherein R₁ is as defined in the structure of Formula IA.

In some embodiments, the compound of Formula IE is represented by Compounds 47-48:

TABLE 2

| Compound's | |
|---|---|
| Number | R₁ of Formula IE |
| 47 | 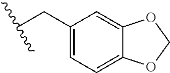 [SMILES: *Cc1ccc2c(c1)OCO2] |
| 48 | 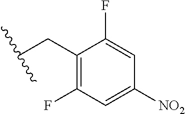 [SMILES: *Cc1c(F)cc([N+](=O)[O-])cc1F] |

In one aspect, the present invention provides a compound represented by the structure of Formula IF:

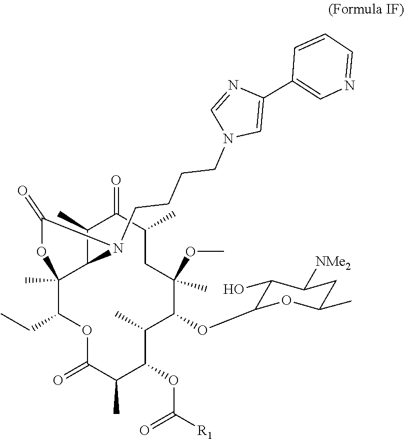

wherein R₁ is as defined in the structure of Formula IA.

In some embodiments, the compound of Formula IF is represented by Compounds 55-56:

TABLE 3

| Compound's | |
|---|---|
| Number | R₁ of Formula IF |
| 55 | 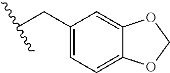 [SMILES: *Cc1ccc2c(c1)OCO2] |
| 56 | 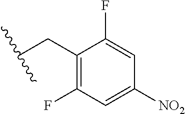 [SMILES: *Cc1c(F)cc([N+](=O)[O-])cc1F] |

In one aspect, the present invention provides a compound represented by the structure of Formula IG:

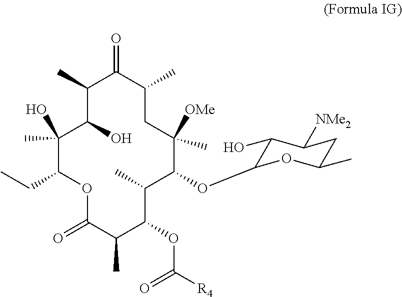

wherein
R₄ is

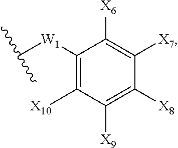

alkyl, alkene, aryl, heteroaryl, -alkylene-N₃, alkylene-cycloalkyl, -alkylene-heterocycloalkyl-aryl, -alkylene-heterocycloalkyl, -alkylene-heteroaryl, -alkylene-heteroaryl-aryl, -alkylene-heteroaryl-alkylene-heterocycloalkyl, -alkylene-aryl-alkylene-aryl, -alkylene-heteroaryl-alkylene-aryl, or -alkylene-heteroaryl-alkylene-heteroaryl;
X₆-X₁₀ are each independently selected from H, halo, CN, OH, NO₂, O-alkylene-aryl, —O-alkylene-heteroaryl, —N-alkylene-aryl, —N-alkylene-heteroaryl, —SO₂— alkylene-aryl, and —SO₂— alkylene-heteroaryl; or two of X₆-X₁₀ are connected via —O—(CH₂)ₘ—O—;
m is an integer from 1-10; and
W₁ is a bond, alkylene, alkenylene, alkynylene, cycloalkyl, heterocyclic, aryl, heteroaryl, or an ether group.

In other aspects, the present invention provides a compound represented by the structure of Formula IG, wherein R₄ is not substituted thiophen or —CH₂-thiophen. In other aspects X₆-X₁₀ is not chloride (Cl). In other aspects wherein at least one of X₆-X₁₀ is not hydrogen. In other aspects if X₆ and X₁₀ are F, then at least one of X₇-X₉ is not H. In other aspects if X₆ and X₁₀ are F, then at least one of X₇-X₉ is not F. In other aspects X₈ is not NO₂.

In other aspects, the present invention provides a compound represented by the structure of Formula IG, wherein:
R₄ is

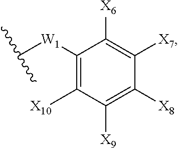

alkylene-N₃, -alkylene-heterocycloalkyl-aryl, -alkylene-heterocycloalkyl, -alkylene-heteroaryl-aryl, -alkylene-heteroaryl-alkylene-heterocycloalkyl, -alkylene-heteroaryl-alkylene-aryl;
X₆-X₁₀ are each independently selected from H, F, I, Br, CN, OH, O— alkylene-aryl, —O— alkylene-heteroaryl, —SO₂-alkylene-aryl, —SO₂-alkylene-heteroaryl; wherein at least one of X₆-X₁₀ is not hydrogen;
wherein if X₆ and X₁₀ are F, then at least one of X₇-X₉ is not H or F; and
W₁ is an alkylene.

In some embodiments the compound of Formula IG is represented by Compounds 2-14, 16, and 18-24:

TABLE 4

| Compound's | |
|---|---|
| Number | R₄ of Formula IG |
| 2 | 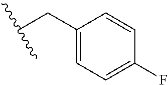 [SMILES: *Cc1ccc(F)cc1] |
| 3 | 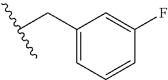 [SMILES: *Cc1cccc(F)c1] |
| 4 | 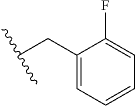 [SMILES: *Cc1ccccc1F] |
| 5 | 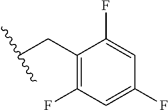 [SMILES: *Cc1c(F)cc(F)cc1F] |
| 6 | 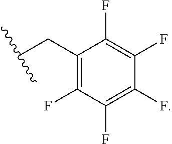 [SMILES: *Cc1c(F)c(F)c(F)c(F)c1F] |
| 7 | 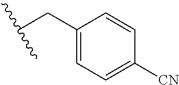 [SMILES: *Cc1ccc(C#N)cc1] |
| 8 | 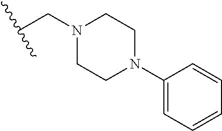 [SMILES: *CN1CCN(c2ccccc2)CC1] |
| 9 | 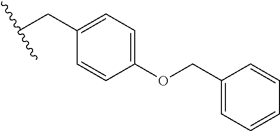 [SMILES: *Cc1ccc(OCc2ccccc2)cc1] |
| 10 | 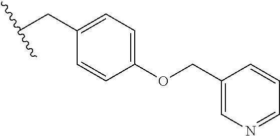 [SMILES: *Cc1ccc(OCc2cccnc2)cc1] |
| 11 | 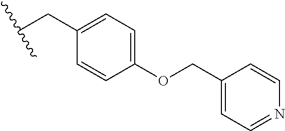 [SMILES: *Cc1ccc(OCc2ccncc2)cc1] |
| 12 | 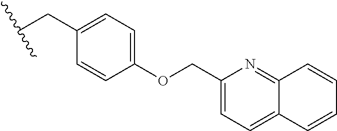 [SMILES: *Cc1ccc(OCc2ccc3ccccc3n2)cc1] |
| 13 | 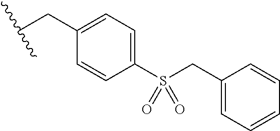 [SMILES: *Cc1ccc(S(=O)(=O)Cc2ccccc2)cc1] |
| 14 | 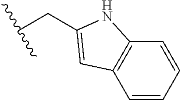 [SMILES: *Cc1cc2ccccc2[nH]1] |
| 16 | 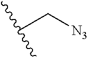 [SMILES: *CN=[N+]=[N-]] |
| 18 | 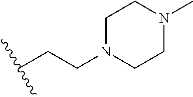 [SMILES: *CCN1CCN(C)CC1] |
| 19 | 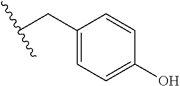 [SMILES: *Cc1ccc(O)cc1] |
| 20 | 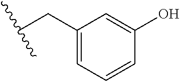 [SMILES: *Cc1cccc(O)c1] |
| 21 | 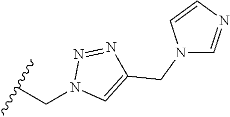 [SMILES: *Cn1cc(Cn2ccnc2)nn1] |
| 22 | 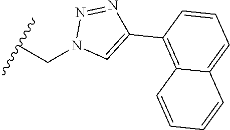 [SMILES: *Cn1cc(-c2cccc3ccccc23)nn1] |
| 23 | 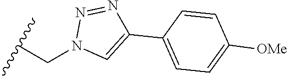 [SMILES: *Cn1cc(-c2ccc(OC)cc2)nn1] |
| 24 | 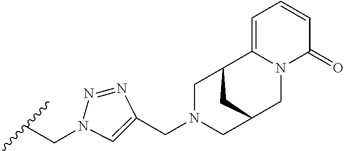 [SMILES: *Cn1cc(CN2C[C@@H]3C[C@H](C2)c2cccc(=O)n2C3)nn1] |

In other embodiments, the alkyl, alkylene, aryl, heteroaryl, cycloalkyl, heterocycloalkyl, alkenylene, alkynylene, heterocyclic, of the compounds of Formula IA, IB, IC, ID, IE, IF and IG are substituted or unsubstituted.

In some embodiments, R₁ of compound of Formula IA, IB, ID, IE, IF is

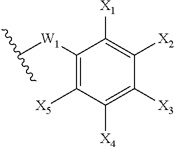

alkyl, alkene, alkylene-N₃, alkylene-cycloalkyl, -alkylene-heterocycloalkyl-aryl, -alkylene-heterocycloalkyl, or -alkylene-heteroaryl. In other embodiments, R₁ is

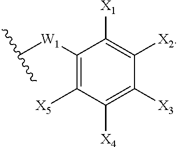

In other embodiments, R₁ is substituted or unsubstituted alkyl. In other embodiments, R₁ is substituted or unsubstituted alkene. In other embodiments, R₁ is substituted or unsubstituted aryl. In other embodiments, R₁ is substituted or unsubstituted heteroaryl. In other embodiments, R₁ is substituted or unsubstituted alkylene-N₃. In other embodiments, R₁ is substituted or unsubstituted alkylene-cycloalkyl. In other embodiments, R₁ is substituted or unsubstituted alkylene-heterocycloalkyl-aryl. In other embodiments, R₁ is substituted or unsubstituted alkylene-heterocycloalkyl. In other embodiments, R₁ is substituted or unsubstituted alkylene-heteroaryl.

In some embodiments, X₁-X₅ of compound of Formula IA, IB, IC, ID, IE, IF each is independently selected from H, halo, CN, NO₂, OH, —O— alkylene-aryl, —O— alkylene-heteroaryl, —N— alkylene-aryl, —N— alkylene-heteroaryl, —SO₂— alkylene-aryl, and —SO₂— alkylene-heteroaryl; or two of X₁-X₅ are connected via —O—(CH₂)ₘ—O—. In other embodiments, X₁-X₅ each is independently H. In other embodiments, X₁-X₅ each is independently halo. In other embodiments, X₁-X₅ each is independently CN. In other embodiments, X₁-X₅ each is independently NO₂. In other embodiments, X₁-X₅ each is independently OH. In other embodiments, X₁-X₅ each is independently substituted or unsubstituted —O-alkylene-aryl. In other embodiments, X₁-X₅ each is independently substituted or unsubstituted —O-alkylene-heteroaryl. In other embodiments, X₁-X₅ each is independently substituted or unsubstituted —N-alkylene-aryl. In other embodiments, X₁-X₅ each is independently substituted or unsubstituted —N— alkylene-heteroaryl. In other embodiments, X₁-X₅ each is independently substituted or unsubstituted —SO₂-alkylene-aryl. In other embodiments, X₁-X₅ each is independently substituted or unsubstituted —SO₂— alkylene-heteroaryl. In other embodiments, two of X₁-X₅ are connected via —O—(CH₂)ₘ—O—, wherein m is an integer from 1-10. In other embodiments m is 1. In other embodiments m is 2. In other embodiments m is 3. In other embodiments m is 4. In other embodiments m is 5. In other embodiments m is 6. In other embodiments m is 7. In other embodiments m is 8, 9 or 10.

In some embodiments, W₁ of Formula IA, IB, ID, IE, IF, IG is a bond, alkylene, alkenylene, alkynylene, cycloalkyl, heterocyclic, aryl, heteroaryl, or an ether group. In other embodiments, W₁ is a bond. In other embodiments, W₁ is substituted or unsubstituted alkylene. In other embodiments, W₁ is substituted or unsubstituted alkenylene. In other embodiments, W₁ is substituted or unsubstituted alkynylene. In other embodiments, W₁ is substituted or unsubstituted cycloalkyl. In other embodiments, W₁ is substituted or unsubstituted heterocyclic. In other embodiments, W₁ is substituted or unsubstituted aryl. In other embodiments, W₁ is substituted or unsubstituted heteroaryl. In other embodiments, W₁ is an ether group (i.e. substituted or unsubstituted alkylene-O-alkylene; or alkylene-O-aryl; or aryl-O-aryl).

In some embodiments, R₂ of Formula IA is H or

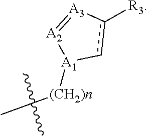

In other embodiments R₂ is H. In other embodiments R₂ is

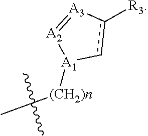

In other embodiments, R₂ is

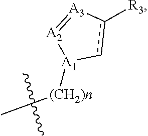

n is an integer from 0-10. In other embodiments, R₂ is

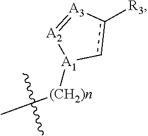

n is 0. In other embodiments, R₂ is

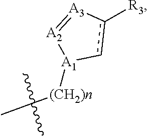

n is 1. In other embodiments, R₂ is

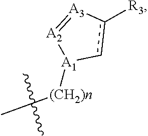

n is 2. In other embodiments, R₂ is

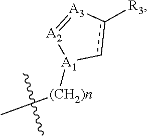

n is 3. In other embodiments, R₂ is

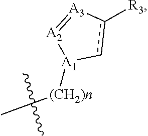

n is 4. In other embodiments, R₂ is

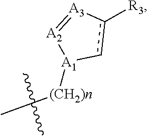

n is 5, 6, 7, 8, 9 or 10. In other embodiments, R₂ is

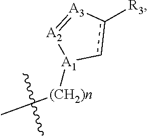

n is an integer from 0-10 and R₃ is substituted or unsubstituted heteroaryl. In other embodiments, R₂ is

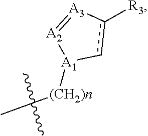

n is an integer from 0-10 and R₃ is substituted or unsubstituted aryl.

In other embodiments, R₂ is

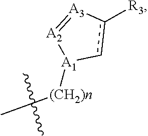

n is an integer from 0-10, R₃ is substituted or unsubstituted heteroaryl or aryl and A₁, A₂, and A₃ are each independently N, NH, NR′, C, CH, CR″, CHR″, CR″R′″ or CH₂. In other embodiments, A₁, A₂, and A₃ are each independently N. In other embodiments, A₁, A₂, and A₃ are each independently NH. In other embodiments, A₁, A₂, and A₃ are each independently NR′. In other embodiments, A₁, A₂, and A₃ are each independently C. In other embodiments, A₁, A₂, and A₃ are each independently CH. In other embodiments, A₁, A₂, and A₃ are each independently CR″. In other embodiments, A₁, A₂, and A₃ are each independently CHR″. In other embodiments, A₁, A₂, and A₃ are each independently CR″R′″. In other embodiments, A₁, A₂, and A₃ are each independently CH₂.

In other embodiments, R₂ is

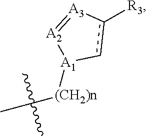

n is an integer from 0-10, R₃ is substituted or unsubstituted heteroaryl or aryl and A₁, A₂, and A₃ are each independently N, NH, NR′, C, CH, CR″, CHR″, CR″R′″ or CH₂; wherein R′, R″, and R′″ are each independently selected from substituted or unsubstituted alkyl, cycloalkyl, aryl, heteroaryl, NO₂, CN, hydroxyl, and halo. In other embodiments, R′, R″, and R′″ are each independently substituted or unsubstituted alkyl. In other embodiments, R′, R″, and R′″ are each independently substituted or unsubstituted cycloalkyl. In other embodiments, R′, R″, and R′″ are each independently substituted or unsubstituted aryl. In other embodiments, R′, R″, and R′″ are each independently substituted or unsubstituted heteroaryl. In other embodiments, R′, R″, and R′″ are each independently NO₂. In other embodiments, R′, R″, and R′″ are each independently CN. In other embodiments, R′, R″, and R′″ are each independently hydroxyl. In other embodiments, R′, R″, and R′″ are each independently halo.

In other embodiments A₁, A₂, and A₃ of Formula ID are each independently N, NH, NR′, C, CH, CR″, CHR″, CR″R′″ or CH₂. In other embodiments, A₁, A₂, and A₃ are each independently N. In other embodiments, A₁, A₂, and A₃ are each independently NH. In other embodiments, A₁, A₂, and A₃ are each independently NR′. In other embodiments, A₁, A₂, and A₃ are each independently C. In other embodiments, A₁, A₂, and A₃ are each independently CH. In other embodiments, A₁, A₂, and A₃ are each independently CR″. In other embodiments, A₁, A₂, and A₃ are each independently CHR″. In other embodiments, A₁, A₂, and A₃ are each independently CR″R′″. In other embodiments, A₁, A₂, and A₃ are each independently CH₂. In some embodiments R′, R″, and R′″ are each independently selected from substituted or unsubstituted alkyl, cycloalkyl, aryl, heteroaryl, NO₂, CN, hydroxyl, and halo. In other embodiments, R′, R″, and R′″ are each independently substituted or unsubstituted alkyl. In other embodiments, R′, R″, and R′″ are each independently substituted or unsubstituted cycloalkyl. In other embodiments, R′, R″, and R′″ are each independently substituted or unsubstituted aryl. In other embodiments, R′, R″, and R′″ are each independently substituted or unsubstituted heteroaryl. In other embodiments, R′, R″, and R′″ are each independently NO₂. In other embodiments, R′, R″, and R′″ are each independently CN. In other embodiments, R′, R″, and R′″ are each independently hydroxyl. In other embodiments, R′, R″, and R′″ are each independently halo.

In some embodiments R₄ of Formula IG is

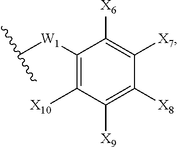

alkyl, alkene, aryl, heteroaryl, alkylene-N₃, alkylene-cycloalkyl, alkylene-heterocycloalkyl-aryl, alkylene-heterocycloalkyl, alkylene-heteroaryl, -alkylene-heteroaryl-aryl, -alkylene-heteroaryl-alkylene-heterocycloalkyl, -alkylene-heteroaryl-alkylene-aryl, -alkylene-aryl-alkylene-aryl, or -alkylene-heteroaryl-alkylene-heteroaryl. In other embodiments R₄ is

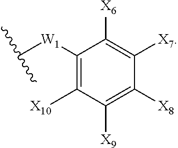

In other embodiments R₄ is substituted or unsubstituted alkyl. In other embodiments R₄ is substituted or unsubstituted aryl. In other embodiments R₄ is substituted or unsubstituted alkene. In other embodiments R₄ is substituted or unsubstituted heteroaryl. In other embodiments R₄ is substituted or unsubstituted alkylene-N₃. In other embodiments R₄ is substituted or unsubstituted alkylene-cycloalkyl. In other embodiments R₄ is substituted or unsubstituted alkylene-heterocycloalkyl-aryl. In other embodiments R₄ is substituted or unsubstituted alkylene-heterocycloalkyl. In other embodiments R₄ is substituted or unsubstituted alkylene-heteroaryl. In other embodiments R₄ is substituted or unsubstituted -alkylene-heteroaryl-aryl. In other embodiments R₄ is substituted or unsubstituted -alkylene-heteroaryl-alkylene-heterocycloalkyl. In other embodiments R₄ is substituted or unsubstituted -alkylene-heteroaryl-alkylene-aryl. In other embodiments R₄ is substituted or unsubstituted -alkylene-aryl-alkylene-aryl. In other embodiments R₄ is substituted or unsubstituted -alkylene-heteroaryl-alkylene-heteroaryl.

In some embodiments X₆-X₁₀ of Formula IG are each independently selected from H, halo, CN, OH, NO₂, O-alkylene-aryl, —O-alkylene-heteroaryl, —N-alkylene-aryl, —N-alkylene-heteroaryl, —SO₂— alkylene-aryl, and —SO₂— alkylene-heteroaryl; or two of X₆-X₁₀ are connected via —O—(CH₂)ₘ—O—; wherein m is an integer from 1-10. In some embodiments X₆-X₁₀ are each independently H. In some embodiments X₆-X₁₀ are each independently halo. In some embodiments X₆-X₁₀ are each independently CN. In some embodiments X₆-X₁₀ are each independently OH. In some embodiments X₆-X₁₀ are each independently NO₂. In some embodiments X₆-X₁₀ are each independently substituted or unsubstituted O-alkylene-aryl. In some embodiments X₆-X₁₀ are each independently substituted or unsubstituted —O-alkylene-heteroaryl. In some embodiments X₆-X₁₀ are each independently substituted or unsubstituted —N-alkylene-aryl. In some embodiments X₆-X₁₀ are each independently substituted or unsubstituted —N-alkylene-heteroaryl. In some embodiments X₆-X₁₀ are each independently substituted or unsubstituted —SO₂-alkylene-aryl. In some embodiments X₆-X₁₀ are each independently substituted or unsubstituted —SO₂-alkylene-heteroaryl. In some embodiments, two of X₆-X₁₀ are connected via —O—(CH₂)ₘ—O—; wherein m is an integer from 1-10. In another embodiment m is 1. In another embodiment m is 2. In another embodiment m is 3. In another embodiment m is 4.

In some embodiments, the term “alkyl” is meant to refer to a saturated hydrocarbon group which is straight-chained or branched. Example alkyl groups include methyl (Me), ethyl (Et), propyl (e.g., n-propyl and isopropyl), butyl(e.g., n-butyl, isobutyl, t-butyl), pentyl (e.g., n-pentyl, isopentyl, neopentyl), and the like. An alkylgroup can contain from 1 to about 20, from 2 to about 20, from 1 to about 10, from 1 to about 8, from 1 to about 6, from 1 to about 4, or from 1 to about 3 carbon atoms.

As used herein, in some embodiments, the term “alkenyl” refers to an unsaturated monovalent chain of carbon atoms including at least one double bond, which may be optionally branched. It is understood that in embodiments that include alkenyl, illustrative variations of those embodiments include lower alkenyl, such as C₂-C₆, C₂-C₄ alkenyl, and the like.

As used herein, in some embodiments, the term “alkynyl” refers to an unsaturated monovalent chain of carbon atoms including at least one triple bond, which may be optionally branched. It is understood that in embodiments that include alkynyl, illustrative variations of those embodiments include lower alkynyl, such as C₂-C₆, C₂-C₄ alkynyl, and the like.

In some embodiments, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

In some embodiments, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms. In some embodiments, “aryl” may be optionally substituted at any one or more positions.

In some embodiments, “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms. In some embodiments, “heteroaryl” may be optionally substituted at any one or more positions capable of bearing a hydrogen atom.

In some embodiments, “heterocycloalkyl” refers to a non-aromatic heterocycle where one or more of the ring-forming atoms are a heteroatom such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. Also included in the definition of heterocycloalkyl are moieties where one or more ring-forming atoms are substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, the term “alkylene” refers to a straight or branched saturated divalent chain of carbon atoms, which may be optionally branched. It is understood that in embodiments that include alkylene, illustrative variations of those embodiments include, but are not limited to, C₁-C₁₂ alkylene, C₁-C₈ alkylene, C₁—C alkylene, C₁-C₄ alkylene, C₁-C₃ alkylene, C₁-C₂ alkylene, C₁ alkylene. Representative alkylene groups include, but are not limited to, methylene, ethylene, propylene, n-butylene, ethenylene, propenylene, n-butenylene, propynylene, n-butynylene, and the like.

As used herein, “alkenylene” refers to a straight or branched divalent aliphatic hydrocarbon group, in certain embodiments having from 2 to about 20 carbon atoms and at least one double bond, e.g., having 2 to 6 carbons, 3 to 4 carbon atoms.

As used herein, “alkynylene” refers to a straight or branched divalent aliphatic hydrocarbon group, in one embodiment having from 2 to about 20 carbon atoms and at least one triple bond, e.g., having 2 to 6 carbons, or 3 to 4 carbons, or 2 to 12 carbons.

In some embodiments, each of the alkyl, cycloalkyl, alkenyl, alkene, cycloalkenyl, alkylene, alkenylene, alkynylene, aryl, heterocycloalkyl, heteroaryl is substituted or unsubstituted with one or more substituents independently selected from alkyl, haloalkyl, hydroxyalkyl, aminoalkyl, aryl, hydroxy, alkoxy, acyloxy, halo, azide, nitro, amino, cyano, alkylamino, dialkylamino, acylamino, thio, carboxylic acid and derivatives thereof, including esters, amides, and nitrites, and combinations thereof.

In each of the foregoing and each of the following embodiments, it is to be understood that the formulas also include any and all hydrates, salts and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates, salts and/or solvates.

In another aspect, the present invention provides a pharmaceutical composition comprising a compound of the invention as described herein, for example, a compound represented by the structure of Formula IA, Formula IB, Formula IC, Formula ID, or Formula IE, or Formula IF or any one of compounds 29-42, 47-48, and 55-56, and a pharmaceutically acceptable carrier.

In another aspect, the present invention provides a pharmaceutical composition comprising a compound of the invention as described herein, for example, a compound represented by the structure of Formula IG or any one of compounds 2-14, 16, and 18-24, and a pharmaceutically acceptable carrier.

The term “pharmaceutically acceptable carrier” refers to a pharmaceutically acceptable material, such as a liquid or solid filler, diluent, excipient, or solvent, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A therapeutically effective dose of a compound of the invention is used, in addition to pharmaceutically acceptable carriers, diluents and/or adjuvants for producing a pharmaceutical composition. The dose of the active compound can vary depending on the route of administration, the age and weight of the patient, the nature and severity of the diseases to be treated, and similar factors. The daily dose can be given as a single dose, which is to be administered once, or be subdivided into two or more daily doses.

Suitable administration forms are oral, parenteral, intravenous, transdermal, topical, inhalative, intranasal and sublingual preparations. Particular preference is given to using oral, parenteral, e.g., intravenous or intramuscular, intranasal, e.g., dry powder or sublingual preparations of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavorings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators. Examples of such additives are tartrate and citrate buffers, ethanol and sequestering agents (such as ethylenediaminetetraacetic acid and its nontoxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or watermiscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g., Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can be presented as powders, solutions or suspensions. Preferably, inhalable preparations are in the form of powders, e.g., as a mixture of the active ingredient with a suitable formulation aid such as lactose.

The preparations are produced, aliquoted and sealed under the customary antimicrobial and aseptic conditions.

In some embodiments, the pharmaceutical composition of the invention is for use in inhibiting selectively S. aureus resistant strains. In some embodiments, the strain is DSM20231, BAA976, or BAA977. In other embodiments, the strain is DSM20231. In some embodiments, the strain is BAA976. In some embodiments, the strain is BAA977. In some embodiments, the compound is a compound of Formula IA-IG. In some embodiments, the compound is any one of compounds 2-14, 16, 18-24, 29-42, 47-48, 55-56. In certain embodiments, the pharmaceutical composition of the invention is for use in inhibiting selectively S. aureus resistant strains, wherein the strain is DSM20231, BAA976, or BAA977, and wherein the compound is a compound of Formula IA.

In other embodiments, the pharmaceutical composition of the invention is for use in inhibiting selectively S. aureus resistant strains, wherein the strain is DSM20231, BAA976, or BAA977, and wherein the compound is any one of compounds 29-42, 47-48, and 55-56.

In some embodiments, the pharmaceutical composition of the invention is for use in inhibiting selectively E. coli strain. In some embodiments, the strain is tolC; MB5747. In some embodiments, the compound is a compound of Formula IA-IG. In other embodiments, the compound is any one of compounds 2-14, 16, 18-24, 29-42, 47-48, 55-56.

In certain embodiments, the pharmaceutical composition of the invention is for use in inhibiting selectively of E. coli strain, wherein the strain is tolC; MB5747, and wherein the compound is a compound of Formula IA.

In other embodiments, the pharmaceutical composition of the invention is for use in inhibiting selectively E. coli strain, wherein the strain is tolC; MB5747, and wherein the compound is any one of compounds 29-42, 47-48, and 55-56.

In some embodiments, the pharmaceutical composition of the invention is for use in the treatment of an infection. In some embodiments, the infection is due to S. aureus resistant strains. In some embodiments, the strain is DSM20231, BAA976, or BAA977. In other embodiments, the strain is DSM20231. In some embodiments, the strain is BAA976. In some embodiments, the strain is BAA977. In some embodiments, the compound is a compound of Formula IA-IG. In some embodiments, the compound is any one of compounds 2-14, 16, 18-24, 29-42, 47-48, 55-56.

In some embodiments, the pharmaceutical composition of the invention is for use in the treatment of an infection, wherein the infection is due to S. aureus resistant strains, and wherein the strain is DSM20231, BAA976, or BAA977. In some embodiments, the strain is DSM20231. In some embodiments, the strain is BAA976. In some embodiments, the strain is BAA977. In some embodiments, the compound is a compound of Formula IA. In some embodiments, the compound is any one of compounds 29-42, 47-48, and 55-56.

In some embodiments, the pharmaceutical composition of the invention is for use in the treatment of an infection. In some embodiments, the infection is due to E. coli strain. In some embodiments, the strain is tolC; MB5747. In some embodiments, the compound is a compound of Formula IA-IG. In other embodiments, the compound is any one of compounds 2-14, 16, 18-24, 29-42, 47-48, 55-56.

In some embodiments, the pharmaceutical composition of the invention is for use in the treatment of an infection, wherein the infection is due to E. coli strain, and wherein the strain is tolC; MB5747. In some embodiments, the compound is a compound of Formula IA. In other embodiments, the compound is any one of compounds 29-42, 47-48, and 55-56.

In yet another aspect, the present invention provides a pharmaceutical composition comprising a compound of the invention as described herein, for example, a compound represented by the structure of Formula IG, or any one of compounds 2-14, 16, and 18-24, and a pharmaceutically acceptable carrier.

In some embodiments, the pharmaceutical composition of the invention is for use in inhibiting selectively S. aureus resistant strains. In some embodiments, the strain is DSM20231, BAA976, or BAA977. In some embodiments, the strain is DSM20231. In some embodiments, the strain is BAA976. In some embodiments, the strain is BAA977. In some embodiments, the compound is a compound of Formula IG. In some embodiments, the compound is any one of compounds 2-6, 8, and 19.

In certain embodiments, the pharmaceutical composition of the invention is for use in inhibiting selectively S. aureus resistant strains, wherein the strain is DSM20231, BAA976, or BAA977, and wherein the compound is a compound of Formula IG.

In certain embodiments, the pharmaceutical composition of the invention is for use in inhibiting selectively S. aureus resistant strains, wherein the strain is DSM20231, BAA976, or BAA977, and wherein the compound is any one of compounds 2-6, 8, and 19.

In some embodiments, the pharmaceutical composition of the invention is for use in inhibiting selectively E. coli strain. In some embodiments, the strain is tolC; MB5747. In some embodiments, the compound is a compound of Formula IG. In some embodiments, the compound is any one of compounds 2-7, 10-11, 13, 16, 19, 21, and 23.

In certain embodiments, the pharmaceutical composition of the invention is for use in inhibiting selectively E. coli strain, wherein the strain is tolC; MB5747, and wherein the compound is a compound of Formula IG.

In certain embodiments, the pharmaceutical composition of the invention is for use in inhibiting selectively E. coli strain, wherein the strain is tolC; MB5747, and wherein the compound is any one of compounds 2-7, 10-11, 13, 16, 19, 21, and 23.

In some embodiments, the pharmaceutical composition of the invention is for use in the treatment of an infection. In some embodiments, the infection is due to S. aureus resistant strains. In some embodiments, the strain is DSM20231, BAA976 or BAA977. In some embodiments, the strain is DSM20231. In some embodiments, the strain is BAA976. In some embodiments, the strain is BAA977. In some embodiments, the compound is a compound of Formula IG. In some embodiments, the compound is any one of compounds 2-6, 8, and 19.

In some embodiments, the pharmaceutical composition of the invention is for use in the treatment of an infection, wherein the infection is due to S. aureus resistant strains, wherein the strain is DSM20231, BAA976 or BAA977, and wherein the compound is a compound of Formula IG.

In certain embodiments, the pharmaceutical composition of the invention is for use in the treatment of an infection, wherein the infection is due to S. aureus resistant strains, wherein the strain is DSM20231, BAA976 or BAA977, and wherein the compound is any one of compounds 2-6, 8, and 19.

In some embodiments, the pharmaceutical composition of the invention is for use in the treatment of an infection. In some embodiments, the infection is due to E. coli strain. In some embodiments, the strain is tolC; MB5747. In some embodiments, the compound is a compound of Formula IG. In some embodiments, the compound is any one of compounds 2-7, 10-11, 13, 16, 19, 21, and 23.

In certain embodiments, the pharmaceutical composition of the invention is for use in the treatment of an infection, wherein the infection is due to E. coli strain, wherein the strain is tolC; MB5747, and wherein the compound is a compound of Formula IG.

In certain embodiments, the pharmaceutical composition of the invention is for use in the treatment of an infection, wherein the infection is due to E. coli strain, wherein the strain is tolC; MB5747, and wherein the compound is any one of compounds 2-7, 10-11, 13, 16, 19, 21, and 23.

In some embodiments, the present invention provides a method for inhibiting selectively S. aureus resistant strains using a pharmaceutical composition of the invention as described herein. In some embodiments, the strain is DSM20231, BAA976, or BAA977. In other embodiments, the strain is DSM20231. In some embodiments, the strain is BAA976. In some embodiments, the strain is BAA977. In some embodiments, the compound is a compound of Formula IA. In some embodiments, the compound is any one of compounds 29-42, 47-48, and 55-56.

In certain embodiments, the present invention provides a method for inhibiting selectively S. aureus resistant strains using a pharmaceutical composition of the invention as described herein, wherein the strain is DSM20231, BAA976, or BAA977, and wherein the compound is a compound of Formula IA.

In other embodiments, the present invention provides a method for inhibiting selectively S. aureus resistant strains using a pharmaceutical composition of the invention as described herein, wherein the strain is DSM20231, BAA976, or BAA977, and wherein the compound is any one of compounds 29-42, 47-48, and 55-56.

In some embodiments, the present invention provides a method for inhibiting selectively E. coli strain using a pharmaceutical composition of the invention as described herein. In some embodiments, the strain is tolC; MB5747. In some embodiments, the compound is a compound of Formula IA. In other embodiments, the compound is any one of compounds 29-42, 47-48, and 55-56.

In certain embodiments, the present invention provides a method for inhibiting selectively E. coli strain using a pharmaceutical composition of the invention as described herein, wherein the strain is tolC; MB5747, and wherein the compound is a compound of Formula IA.

In other embodiments, the present invention provides a method for inhibiting selectively E. coli strain using a pharmaceutical composition of the invention as described herein, wherein the strain is tolC; MB5747, and wherein the compound is any one of compounds 29-42, 47-48, and 55-56.

In some embodiments, the present invention provides a method for the treatment of an infection using a pharmaceutical composition of the invention as described herein. In some embodiments, the infection is due to S. aureus resistant strains. In some embodiments, the strain is DSM20231, BAA976, or BAA977. In some embodiments, the compound is a compound of Formula IA. In other embodiments, the compound is any one of compounds 29-42, 47-48, and 55-56.

In some embodiments, the present invention provides a method for the treatment of an infection using a pharmaceutical composition of the invention as described herein, wherein the infection is due to S. aureus resistant strains, and wherein the strain is DSM20231, BAA976, or BAA977. In some embodiments, the compound is a compound of Formula IA. In other embodiments, the compound is any one of compounds 29-42, 47-48, and 55-56.

In some embodiments, the present invention provides a method for the treatment of an infection using a pharmaceutical composition of the invention as described herein. In some embodiments, the infection is due to E. coli strain. In some embodiments, the strain is tolC; MB5747.

In some embodiments, the compound is a compound of Formula IA. In other embodiments, the compound is any one of compounds 29-42, 47-48, and 55-56.

In some embodiments, the present invention provides a method for the treatment of an infection using a pharmaceutical composition of the invention as described herein, wherein the infection is due to E. coli strain, and wherein the strain is tolC; MB5747. In some embodiments, the compound is a compound of Formula IA. In other embodiments, the compound is any one of compounds 29-42, 47-48, and 55-56.

In some embodiments, the present invention provides a method for inhibiting selectively S. aureus resistant strains using a pharmaceutical composition of the invention as described herein.

In some embodiments, the strain is DSM20231, BAA976, or BAA977. In some embodiments, the strain is DSM20231. In some embodiments, the strain is BAA976. In some embodiments, the strain is BAA977. In some embodiments, the compound is a compound of Formula IG. In some embodiments, the compound is any one of compounds 2-6, 8, and 19.

In some embodiments, the present invention provides a method for inhibiting selectively S. aureus resistant strains using a pharmaceutical composition of the invention as described herein, wherein the strain is DSM20231, BAA976, or BAA977, and wherein the compound is a compound of Formula IG.

In certain embodiments, the present invention provides a method for inhibiting selectively S. aureus resistant strains using a pharmaceutical composition of the invention as described herein, wherein the strain is DSM20231, BAA976, or BAA977, and wherein the compound is any one of compounds 2-6, 8, and 19.

In some embodiments, the present invention provides a method for inhibiting selectively E. coli strain using a pharmaceutical composition of the invention as described herein. In some embodiments, the strain is tolC; MB5747. In some embodiments, the compound is a compound of Formula IG. In some embodiments, the compound is any one of compounds 2-7, 10-11, 13, 16, 19, 21, and 23.

In certain embodiments, the pharmaceutical composition of the invention is for use in inhibiting selectively E. coli strain using a pharmaceutical composition of the invention as described herein, wherein the strain is tolC; MB5747, and wherein the compound is a compound of Formula IG.

In certain embodiments, the pharmaceutical composition of the invention is for use in inhibiting selectively E. coli strain using a pharmaceutical composition of the invention as described herein, wherein the strain is tolC; MB5747, and wherein the compound is any one of compounds 2-7, 10-11, 13, 16, 19, 21, and 23.

In some embodiments, the present invention provides a method for the treatment of an infection using a pharmaceutical composition of the invention as described herein. In some embodiments, the infection is due to S. aureus resistant strains. In some embodiments, the strain is DSM20231, BAA976 or BAA977. In some embodiments, the strain is DSM20231. In some embodiments, the strain is BAA976. In some embodiments, the strain is BAA977. In some embodiments, the compound is a compound of Formula IG. In some embodiments, the compound is any one of compounds 2-6, 8, and 19.

In certain embodiments, the present invention provides a method for the treatment of an infection using a pharmaceutical composition of the invention as described herein, wherein the infection is due to S. aureus resistant strains, wherein the strain is DSM20231, BAA976 or BAA977, and wherein the compound is a compound of Formula IG.

In certain embodiments, the present invention provides a method for the treatment of an infection using a pharmaceutical composition of the invention as described herein, wherein the infection is due to S. aureus resistant strains, wherein the strain is DSM20231, BAA976 or BAA977, and wherein the compound is any one of compounds 2-6, 8, and 19.

In some embodiments, the present invention provides a method for the treatment of an infection using a pharmaceutical composition of the invention as described herein. In some embodiments, the infection is due to E. coli strain. In some embodiments, the strain is tolC; MB5747.

In some embodiments, the compound is a compound of Formula IG. In some embodiments, the compound is any one of compounds 2-7, 10-11, 13, 16, 19, 21, and 23.

In certain embodiments, the present invention provides a method for the treatment of an infection using a pharmaceutical composition of the invention as described herein, wherein the infection is due to E. coli strain, wherein the strain is tolC; MB5747, and wherein the compound is a compound of Formula IG.

In certain embodiments, the present invention provides a method for the treatment of an infection using a pharmaceutical composition of the invention as described herein, wherein the infection is due to E. coli strain, wherein the strain is tolC; MB5747, and wherein the compound is any one of compounds 2-7, 10-11, 13, 16, 19, 21, and 23.

The term “treatment” as used herein refers to the administering of a therapeutic amount of the composition as described herein which is effective to ameliorate undesired diseases, disorders, including symptoms associated with a diseases or disorders, to prevent the manifestation of such diseases, disorders, including symptoms associated with a diseases or disorders before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The following examples are presented in order to more fully illustrate the preferred embodiments of the invention. They should in no way be construed, however, as limiting the broad scope of the invention.

### EXAMPLES

### Example 1: Synthesis of 11,12-Diole Acylides

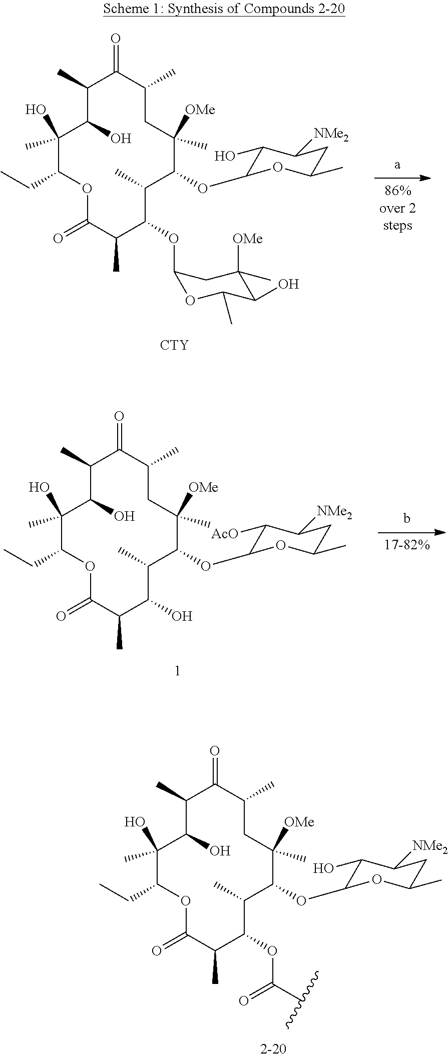

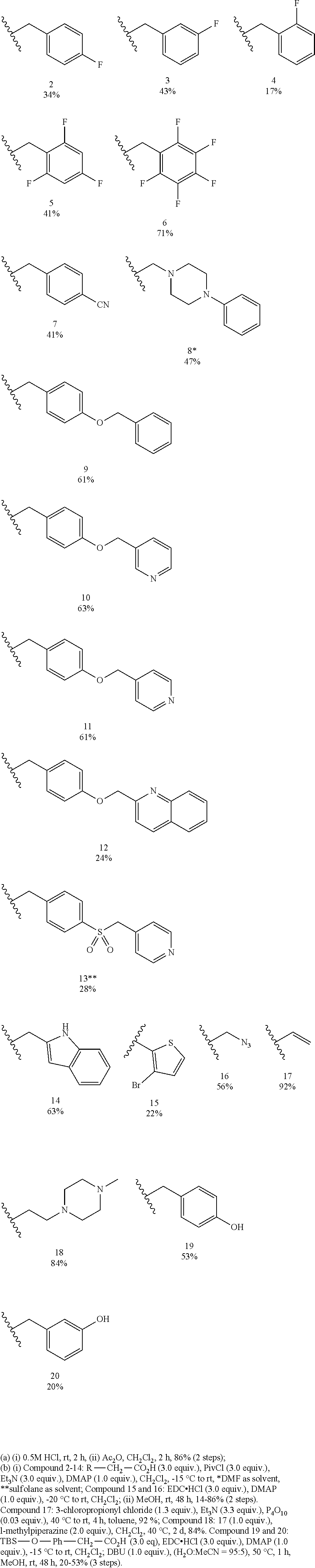

A series of acylides 2-20 was synthesized by 3-O-acylation of 2′-O-acetyl-5-O-desosaminyl-6-O-methylerythronolide A (1) with corresponding acetic acid derivatives. Subsequently, the 2′-O-acetyl group was removed by methanolysis which yielded the target compounds in 14-86% yield (Scheme 1). To this end, in situ prepared mixed anhydrides from pivaloyl chloride (PivCl) and the respective carboxylic acid in CH₂Cl₂ were condensed with 1 in presence of 4-dimethylamino pyridine (DMAP) which allowed the synthesis of compounds 2-14. For the synthesis of compounds 15-16, Steglich conditions using 1-[3-(dimethylamino)propyl]-3-ethylcarbodiimide hydrochloride (EDC-HCl) were employed instead. TBDMS-protected carboxcylic acids were utilized for preparing the phenolic 19-20. Compound 18 was synthesized by 1,4-addition of 1-methylpiperazine to precursor 17. In case of arylethers 9-12, the ether group was introduced prior to acylide formation by Williamson synthesis.

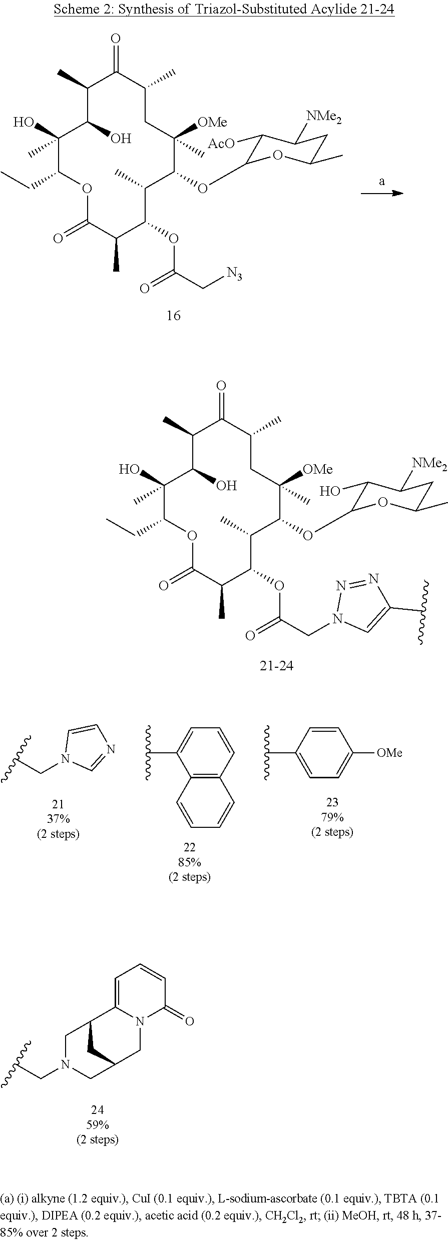

Using 2-azidoacetic acid, compound 16 was synthesized as precursor for Cu-catalyzed azide-alkyne cycloaddition to yield heterocyclic acylides 21-24 with yields between 37-85% over two steps (Scheme 2).

### Example 2: Synthesis of 11,12-Cyclic Carbamate Acylides

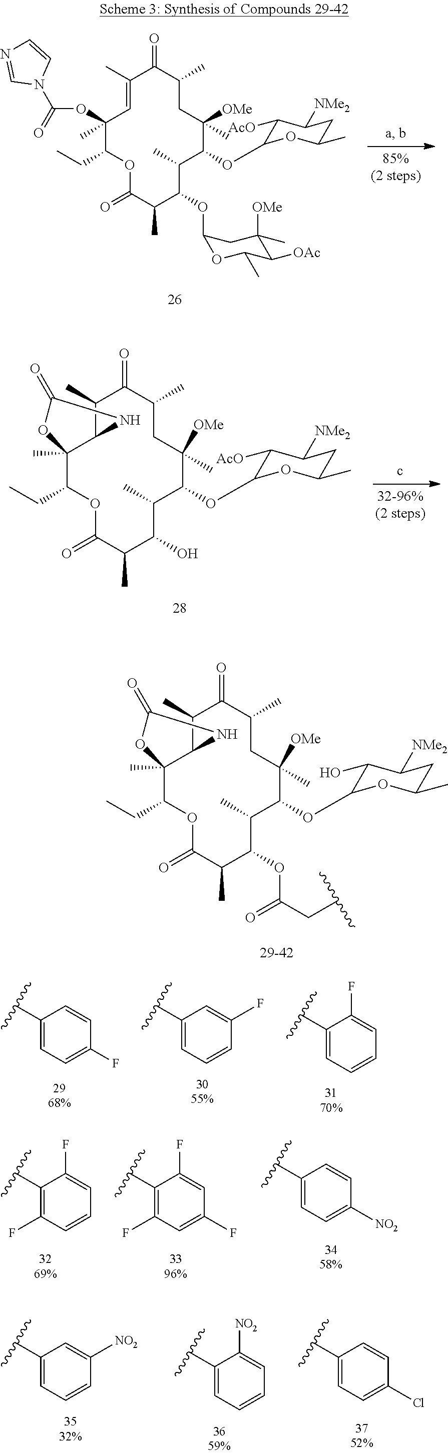

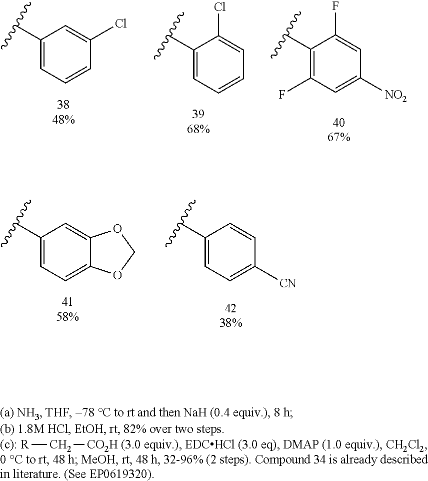

A series of carbamacylides (11,12-carbamate acylides) were synthesized using selected acyl-substituents after preliminary bioactivity screens of 2-24. To enable the desired set of carbamacylides, compound 26 was used to construct the carbamate acylide-precursor 28 with 82% yield over two steps (Scheme 3). Using the abovementioned acylation procedures, carbamacylides 29-42 were prepared with 32-96% yields over two steps.

### Example 3: Synthesis of Ketolide-Acylide Hybrid Compounds

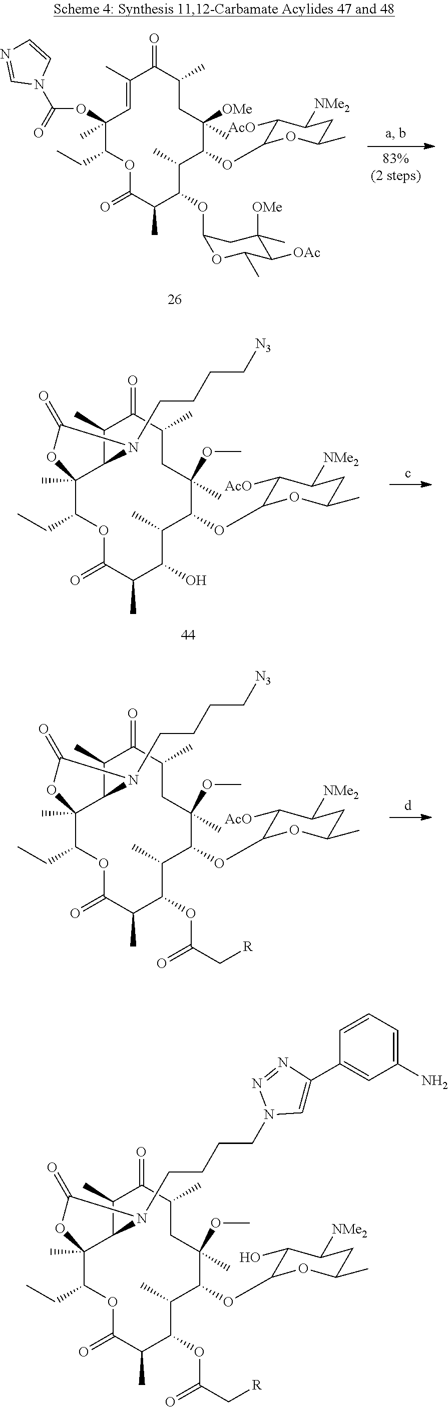

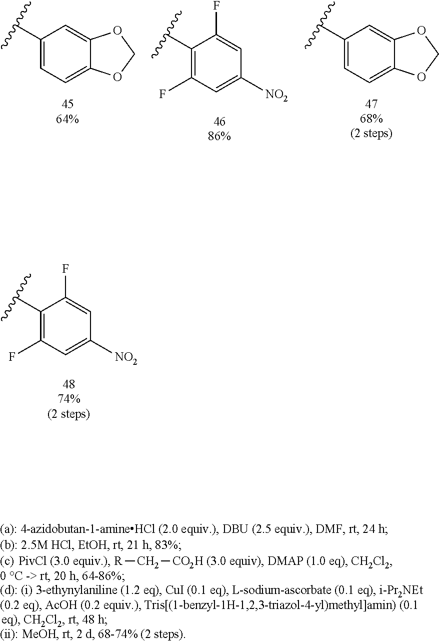

A set of ketolide-acylide hybrid compounds based on the heterocyclic side-chains of solithromycin and telithromycin were synthesized to study potential cooperative effects between modifications at positions 3-O and 11,12-O.

Compound 44 was synthesized and acylated with the most active acylide side chains 3,4-(methylenedioxy)phenylacetic acid and 2,6-difluoro-4-nitro phenylacetic acid, based on our pre-screening, to yield the solithromycin-derived carbamacylides 47 and 48 in 68% and 74% over two steps, respectively (Scheme 4).

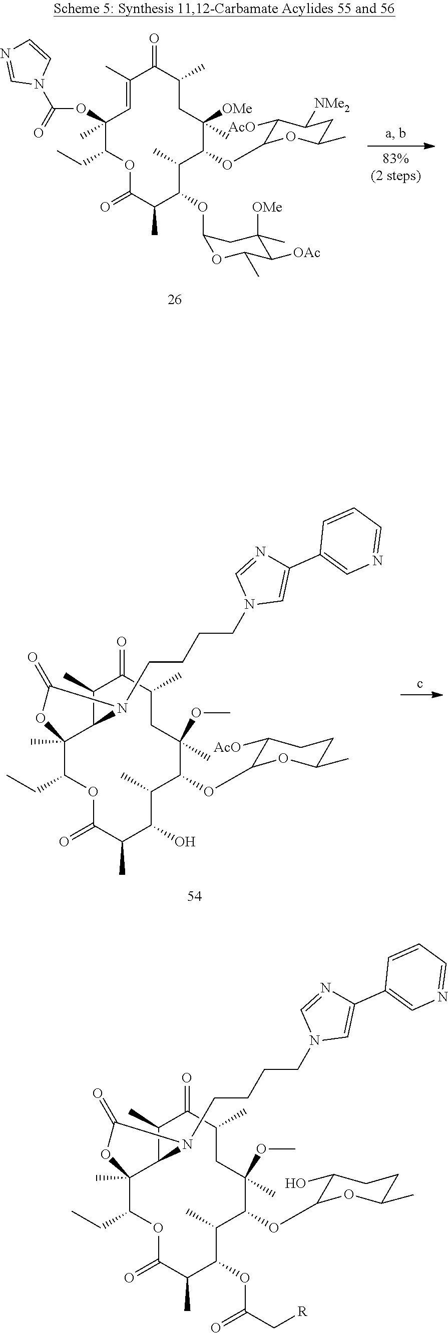

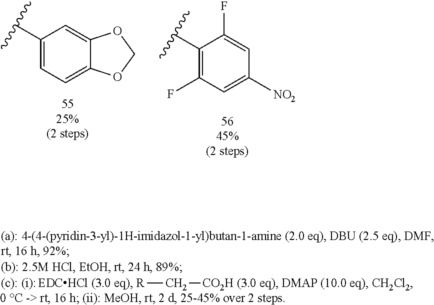

In an analogous fashion, two telithromycin-derived carbamate acylides 55 and 56 were prepared utilizing the previously described carbamacylide-precursor 54 in an overall yield of 25% and 45% over two steps, respectively (Scheme 5).

### Example 4: Biological Testing

Acylides 2-24, as well as carbamate acylides 29-42, 47-48 and 55 and 56 were tested for antibacterial activity against a panel of bacterial isolates, including macrolide-resistant strains. Furthermore, potential toxicity against human HEK293 and red blood cells was assessed. All compounds were submitted to ComPlat (Karlsruhe Institute of Technology, Germany) for compound management and screening. As reference antibiotics, the macrolide antibiotics clarithromycin (CTY), telithromycin (TEL), solithromycin (SOL), as well as the previously reported acylides TEA0777 and X10324 (See, Wayne, P. C. a. L. S. I., Methods for Dilution Antimicrobial Susceptibility Tests for Bacteria That Grow Aerobically. CLSI document M07. CLSI 2009) were employed.

The antibacterial activity was investigated against:
1) S. aureus subsp. aureus Rosenbach 1884 (DSM 20231),
2) S. aureus subsp. Aureus (ATCC® BAA977™) iMLS_{B},
3) S. aureus subsp. aureus (ATCC® BAA976™) msr(A), S. aureus subsp. Aureus (ATCC® 43300™).

Furthermore, the Gram-negative bacteria E. coli (DSM 30083), Acinetobacter baumannii Bouvet and Grimont 1986 (DSM 30007) and Pseudomonas aeruginosa (DSM 50071) were assayed. The compound libraries were tested using clarithromycin (CTY), telithromycin (TEL), solithromycin (SOL) and the previously described acylide antibiotics TEA-0777 and TRMA-121 (X10324; KIT ComPlat) as reference antibiotics. The MIC values were determined as previously described (See, Wayne, P. C. a. L. S. I., Methods for Dilution Antimicrobial Susceptibility Tests for Bacteria That Grow Aerobically. CLSI document M07. CLSI 2009; Albada, H. B., et al., Modulating the activity of short arginine-tryptophan containing antibacterial peptides with N-terminal metallocenoyl groups. 2012. 8(1): p. 1753-1764).

### Results and Discussion

### Antimicrobial Activity of 11,12-Diol Acylides

The biological activity of acylides 2-24 were tested for antibacterial activity.

Furthermore, antifungal activity, cytotoxicity against HEK293 cells and possible hemolytic activity against red blood cells was assayed, all results are listed in Table 1. A majority of the tested acylides show potent antibacterial activity against S. aureus except for the constitutively resistant MRSA-strain ATCC 43300.

Interestingly, it was observed a strong dependence of the minimum inhibitory concentration (MIC) on substituent-positioning of the benzene moiety as in the phenol-derivatives 19 (para-phenol, MIC 4 μg·mL⁻¹/S. aureus BAA976) and 20 (meta-phenol, MIC 32 μg·mL⁻¹/S. aureus BAA976). In contrast, for the corresponding fluoroaromatic compounds 2, 3, and 4 the positioning of the fluorine-substituents does not have a significant influence on the MIC-value.

However, an increasing number of fluorine atoms as in 2,4,6-trifluoro phenyl substituted compound 5, resulted in the highest antibiotic activity (MIC=0.25 μg·mL⁻¹/S. aureus). A fluorine atom can thus replace in this special case the NO₂-functionalization in the previously described reference compound X10324. (Gerhard Konig et al., Rational prioritization strategy allows the design of macrolide derivatives that overcome antibiotic resistance, PNAS, 2021 Vol. 118 No. 46 e2113632118.)

Acylide 5 showed an up to 32-fold increased activity against S. aureus strains with an inducible macrolide resistance in comparison to the clinically used drugs telithromycin and clarithromycin and is comparable to the candidate antibiotic solithromycin. However, the previously described pentafluoro phenyl substituted acylide 6 shows similar activity to the mono fluorine-substituted acylides 2-4.¹⁵ Replacement of the NO₂-group in TEA0777 by the bioisosteric nitrile substituent yielded compound 7 with a significantly decreased biological potency.

The least active acylides were triazole-based 21-24 and furthermore the heterocyclic variants based on methyl piperazine (18), indole (14) and thiazole (15). Compounds with a second ring system (8-13) in the acylide ring motif generally showed a decreased antibiotic activity against Gram-positive strains, however 10-13 reveal an appreciable activity against a tolC-deficient E. coli isolate. The larger ring system may thus prevent membrane passage but not necessarily the inhibition of the bacterial ribosome.

### 11,12-NH-Carbamate Acylides

The activity of 11,12-diol acylides was compared to the 11,12-NH-carbamtes 29-42 (vide supra, Table 2). A majority of the tested NH-carbamate acylides show potent antibacterial activity.

All synthesized NH-carbamates (scheme 3, table 2) show improved antibiotic activities in comparison to clarithromycin, telithromycin and the corresponding 11,12-OH acylides 2-24. In particular, compounds 34, 35, 40, and 41 reveal superior activity against inducible macrolide-resistant S. aureus BAA 976 msr(A) and BAA 977 iMLS_{B}. 40 and 41 break the induction of resistance mechanisms and show indistinguishable activity against both resistant strains and the non-resistant reference strain S. aureus DSM20231. All tested macrolides apart from the clinically used clarithromycin and telithromycin show activity against the Gram-negative tolC-deficient E. coli and furthermore 41 again shows superior activity against the Gram-negative ESKAPE pathogen Acinetobacter baumanii, which cannot be targeted by current macrolide antibiotics.

Substitution of a para-F atom (33) or H-atom (32) to a NO₂-group (40) has a significant effect on the bioactivity against S. aureus BAA 976 msr(A) and BAA 977 iMLS_{B} compared with the reference antibiotics. There is no significant positioning effect in mono substituted acylides 29-39 and 42.

None of the compounds show toxic behavior in the assays against HEK293 and red blood cells.

The introduction of the carbamate at C-11 and C-12 establishes a powerful, easy to accomplish tool to improve acylide bioactivity. Compounds 40 and 41 represent novel macrolide antibiotic structures in which relatively minor modifications on the macrolide core by means of combination of a non-substituted 11,12-NH-carbamate and a cladinose-to-acyl exchange leads to significant biological efficacy.

### Solithromycin- and Telithromycin-Based Hybrids

In solithromycin and telithromycin the alkyl-aryl moiety which was tethered to the carbamate at position 11,12 restores the loss of activity upon removal of the resistance-inducing cladinose. Furthermore, it improved activity against various pathogens, the corresponding sidechain can thus be indicated as a pharmacophoric element. Next, inventors were interested in whether the activity of acylides would benefit from fusions to the side-arms of ketolide antibiotics, possibly beyond the effect of a plain NH-carbamate. However, it would also be plausible that such a pharmacophoric side-arm would distort the acylide-ribosome interaction and thus lead to a loss-of-activity. Furthermore, the very same side-arm might constitute a second binding site at the molecular target, leading to an increased affinity of the ligand.

Hence, acylide ketolide hybrids were synthesized in which the 11,12-carbamate is merged individually with the aryl moieties based on solithromycin and telithromycin. Additionally, 2-desfluoro-solithromycin was synthesized for use as a direct reference compound.¹¹ Testing against the same panel of strains revealed all hybrids as potent antibiotics. As expected, 2-desfluoro-solithromycin (50) is significantly less potent than solithroymcin. The 11,12-NH carbamates 40 and 41, however, exert an effect similar to the hybrids 47 and 48. Hybrid 48, carrying the novel electron-poor benzene derivative, is significantly more active than the reference compound 50, however, its activity is in a similar range to solithromycin itself. The acylide moiety can thus compensate for the loss of activity due to the missing C2-F atom in 50 and can exert its effect independent from the ketolide side-arm.

The combination of different activity-conferring motifs into the macrolide core can serve as a synthetically challenging yet interesting means to interrogate macrolide-ribosome interactions.

In conclusion, this application describes the synthesis of a compound library consisting of 41 acylide antibiotics that belong to the group of 11,12-diol acylides, 11,12-NH-carbamate acylides and ketolide-acylide hybrid macrolides. Most of the synthesized antibiotics were not described to date. Comparison of the three subsets showed a remarkable increase in antibacterial activity when the natural 11,12-diol functionality is transferred into a cyclic-N-carbamate. Specific activity against inducible macrolide-resistant Gram-positive S. aureus strains (BAA977 msr(A) and BAA977 iMLS_{B}) was increased up to 128-fold compared to clarithromycin and telithromycin. Noteworthy, some of our macrolides show activity against the Gram-negative ESKAPE pathogen A. baumannii, which is fully resistant to clinically used macrolids. The addition of two different ketolide side-arms to selected acylides did not further improve their antibiotic activities.

These results illustrate the efficiency of identifying new macrolide antibiotics via the synthetically rapidly accessible acylides, which present themselves as an easily diversifiable class of substances and provide access to resistance-breaking compounds in relatively small libraries.

### Example 5: Structural Studies

### Materials and Methods

### Stability in Human Blood Serum

All compounds were tested for stability inhuman blood serum and found to be completely stable following a protocol by Spork et. Al (A. P. Spork et al., Lead structures for new antibacterials: Stereo controlled synthesis of a bioactive muraymycin analogue. Chemistry 20, 15292-15297 (2014).

### Minimum Inhibitory Concentration (MIC) Assay

In order assess the potency of acylide against resistant strains broth microdilution assays were performed. It was investigated the antibacterial activity against:
S. aureus subsp. aureus Rosenbach 1884 (DSM 20231),
S. aureus subsp. aureus (ATCC® BAA976™) msr(A),
S. aureus subsp. aureus (ATCC® BAA977™) iMLS_{B},
S. aureus subsp. aureus (ATCC® 43300™)

Furthermore, the Gram-negative bacteria E. coli (DSM 30083), Acinetobacter baumannii Bouvet and Grimont 1986 (DSM 30007) and Pseudomonas aeruginosa (DSM50071) were assayed. The compound libraries were tested using clarithromycin, telithromycin, solithromycin and the previously described acylide antibiotics TEA-0777 as reference antibiotics. The MIC values were determined as previously described (Wayne, P. C. a. L. S. I., Methods for Dilution Antimicrobial Susceptibility Tests for Bacteria That Grow Aerobically. CLSI document M07. CLSI 2009).

### Inhibition Assays and IC50 Determination

The inhibition effect of the acylides on S. aureus ribosomes was tested in a bacterial coupled transcription/translation assay system where the expression of the luciferase gene was measured. The luciferase gene was inserted into a plasmid with T7 RNA polymerase promoter. Serial dilution of each oligonucleotide in water (the concentrations of the stock solutions were determined by measuring their absorbance at 260 nm) from 1 mM, to 64 nM were prepared; a reaction mixture including 0.12 v/v E. coli S100 lysate, 300 nM SA30S, 300 nM SA50S, 1.3 mM of amino acids mix, 0.25 mg/ml creatine kinase, 0.027 mg/ml T7 RNA polymerase, 0.003 g/ml luciferase plasmid, 174 g/ml E. coli tRNA mixture, 160 mM Hepes KOH (pH 7.5), 6.5% PEG 8K, 0.074 mg/ml tyrosine, 208 mM potassium glutamate, 1.8 mM dithiothreitol (DTT), 1.3 mM adenosine triphosphate (ATP), 0.86 mM guanosine triphosphate (GTP), 0.86 mM cytidine triphosphate (CTP) and 0.86 mM uridine triphosphate (UTP), 0.663 mM cyclic adenosine monophosphate (cAMP), 83 mM creatine phosphate, 0.036 mg/ml folinic acid, 28 mM ammonium acetate and 14.8 mM magnesium acetate was prepared; 30 μl of the reaction mixture were incubated for 50 min at 37° C. in the presence of different concentrations of the various acylides tested; the reactions were terminated with the addition of 48 μl of water; luciferin assay reagent (LAR, Promega) at 5:3 (luciferase: reaction mix) volume ratio was added to the mixture and photoluminescence was instantly measured; dose/response curves were fitted to the experimental data with GraFit to calculate IC50 values using the 4-parameter equation:

$$y = \frac{d - c}{1 + {( \frac{x}{{IC}_{50}} )}^{s}} + c$$

where x is the concentration of inhibitor, y is the luminescence, c is the lower limit of the curve, d is the upper limit of the curve and s is the slope of the curve.
S. aureus (SA) Growth and Ribosomes Purification

S. aureus strain RN4220 (American Type Culture Collection 35556) was grown and disrupted as described previously and ribosomes were purified as described previously. (Halfon, Y., et al., (2019). Exit tunnel modulation as resistance mechanism of S. aureus erythromycin resistant mutant. Scientific Reports, 9(1), 11460).

### Cryo-EM Data Collection and Refinement of SA50S-Acylides Structures

Ribosome samples (3.5 μL, ~0.4 mg/mL; 70S ribosomes for compounds 40 and 48, 50S subunits for X10324 and 48) were applied to glow-discharged holey carbon grids (Quantifoil R2/2) coated with a continuous thin carbon film. The grids were blotted and plunge frozen using a Vitrobot Mark IV (Thermo-Fischer Scientific). Cryo-EM micrographs were collected at liquid nitrogen temperature on a Titan Krios electron microscope (Thermo-Fischer Scientific) operating at 300 kV. Micrographs were recorded on a Gatan K3 direct electron detector at a nominal magnification of 105K with a pixel size of 0.85 Å/pixel and a dose rate of 1 electrons/Å²/s. Defocus values ranged from −1.5 to −3.5 □m. Movies were patched-framed-motion-corrected and dose-weighted using Motioncor2 (Zheng, S. Q., et al., MotionCor2: anisotropic correction of beam-induced motion for improved cryo-electron microscopy. 2017. 14(4): p. 331-332). CTFFIND-3 (Mindell J A, Grigorieff N. Accurate determination of local defocus and specimen tilt in electron microscopy. J Struct Biol. 2003 June; 142(3):334-47. doi: 10.1016/s1047-8477(03)00069-8. PMID: 12781660) was used for estimation of the contrast transfer function parameters, and RELION-3.1 (Zivanov J, Nakane T, Scheres S H W. Estimation of high-order aberrations and anisotropic magnification from cryo-EM data sets in RELION3.1. IUCrJ. 2020 Feb. 11; 7(Pt 2):253-267. doi: 10.1107/S2052252520000081. PMID: 32148853; PMCID: PMC7055373) for downstream image processing steps. Semi-automatic particle picking was then subjected to unsupervised 3D classification using a cryo-EM map of the S. aureus ribosome) as initial reference (EMD-10079 for 40 and 48; EMID-0243 for X10324 and 56). All classes appearing to contain well-formed ribosomal particles (70S ribosomes for 40 and 48, 50S subunits for X10324 and 56), were selected and used for auto-refinement with RELION 3.1. These particles were then subjected to CTF refinement, particle polishing and 3D-refinement. For 40 and 48, a further multibody refinement step with separate masks for the 50S and the 30S subunits was used to get a 50S map. The resolution of the final 50S maps was 2.45, 2.46, 2.28, 2.34 Å for X10324, 40, 48, and 56, respectively. Average maps resolutions were determined using the gold-standard FSC=0.143 criterion as implemented in RELION 3.1 and M-triage as implemented in Phenix.

### Model Building and Refinement of the SA50S-Acylides Structures

Model building of rRNA and RPs was performed combining template-guided and de novo model building in COOT (Emsley, P., et al., Features and development of Coot. 2010. 66(4): p. 486-501). The coordinates of the S. aureus ribosome (PDB 6HMA) were used as an initial template for model building and were docked onto EM-maps using UCSF Chimera. RNA modifications were manually modeled with coordinates and library files for the modified residues were generated through PHENIX.Elbow (Moriarty, N. W., R. W. Grosse-Kunstleve, and P. D. J. A. C. S. D. B. C. Adams, electronic Ligand Builder and Optimization Workbench (eLBOW): a tool for ligand coordinate and restraint generation. 2009. 65(10): p. 1074-1080). The coordinates and library files for each acylide compound were generated using Grade Web Server (http://grade.globalphasing.org) and were fitted using COOT. Model refinement was performed using an iterative approach including real space refinement and geometry regularization in COOT, followed by real space refinement using the PHENIX suite (Liebschner D, Afonine P V, Baker M L, Bunkóczi G, Chen V B, Croll T I, Hintze B, Hung L W, Jain S, McCoy A J, Moriarty N W, Oeffner R D, Poon B K, Prisant M G, Read R J, Richardson J S, Richardson D C, Sammito M D, Sobolev O V, Stockwell D H, Terwilliger T C, Urzhumtsev A G, Videau L L, Williams C J, Adams P D. Macromolecular structure determination using X-rays, neutrons and electrons: recent developments in Phenix. Acta Crystallogr D Struct Biol. 2019 Oct. 1; 75(Pt 10):861-877. doi: 10.1107/S2059798319011471. Epub 2019 Oct. 2. PMID: 31588918; PMCID: PMC6778852). The final model was validated using MolProbity. USCF Chimera and UCSF ChimeraX (Pettersen E F, Goddard T D, Huang C C, Meng E C, Couch G S, Croll T I, Morris J H, Ferrin T E. UCSF ChimeraX: Structure visualization for researchers, educators, and developers. Protein Sci. 2021 January; 30(1):70-82. doi: 10.1002/pro.3943. Epub 2020 Oct. 22. PMID: 32881101; PMCID: PMC7737788) were used throughout for map and model visualization.

### Studies

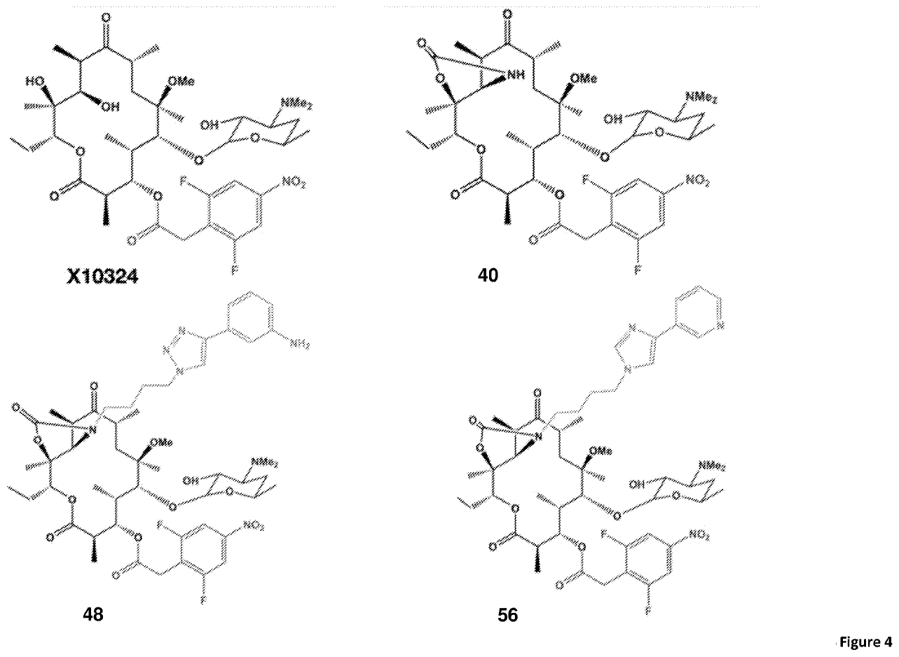

The newly designed acylide compounds X10324, 40, 48 and 56 presented in this study are provided in FIG. 4.

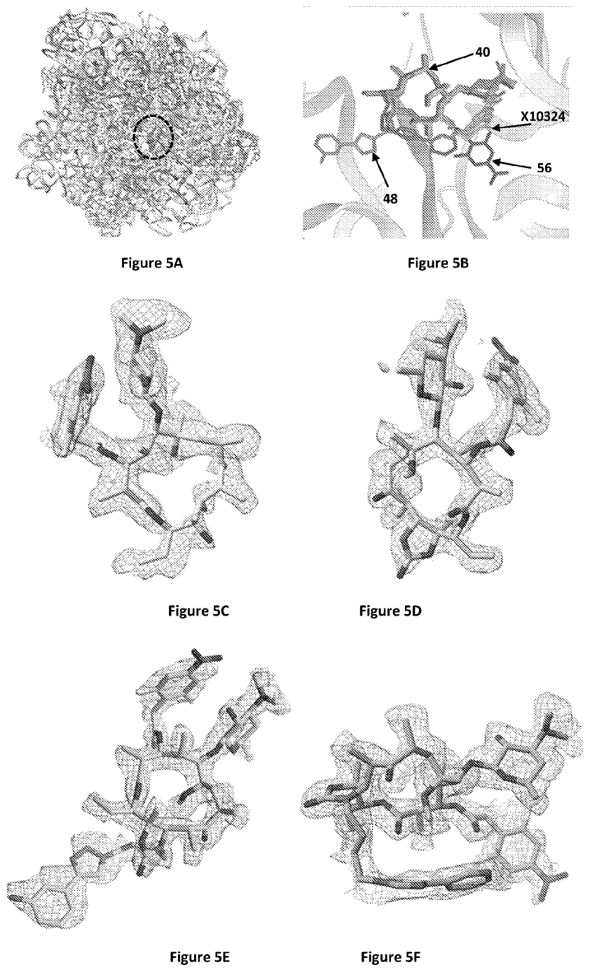

Cryo-EM maps of SA50S in complex with X10324, 40, 48 and 56 were able to be obtained at 2.45 Å, 2.46 Å, 2.28 Å, and 2.34 Å resolution, respectively. The structures indicate that the acylides bind at the upper rim of the nascent peptide exit tunnel (NPET) in close proximity to the PTC (FIG. 5A), in agreement with other known macrolides bound to the large ribosomal subunit (Schlünzen, F., et al., Structural basis for the interaction of antibiotics with the peptidyl transferase centre in eubacteria. Nature, 2001. 413(6858): p. 814; Dunkle, J. A., et al., Structures of the Escherichia coli ribosome with antibiotics bound near the peptidyl transferase center explain spectra of drug action. Proceedings of the National Academy of Sciences, 2010. 107(40): p. 17152-17157; Belousoff, M. J., et al., Crystal structure of the synergistic antibiotic pair, lankamycin and lankacidin, in complex with the large ribosomal subunit. Proceedings of the National Academy of Sciences, 2011. 108(7): p. 2717-2722; Hansen, J. L., et al., The structures of four macrolide antibiotics bound to the large ribosomal subunit. Molecular cell, 2002. 10(1): p. 117-128; Almutairi, M. M., et al., Co-produced natural ketolides methymycin and pikromycin inhibit bacterial growth by preventing synthesis of a limited number of proteins. Nucleic acids research, 2017. 45(16): p. 9573-9582; Hansen, J. L., et al., The structures of four macrolide antibiotics bound to the large ribosomal subunit. Mol Cell, 2002. 10(1): p. 117-28).

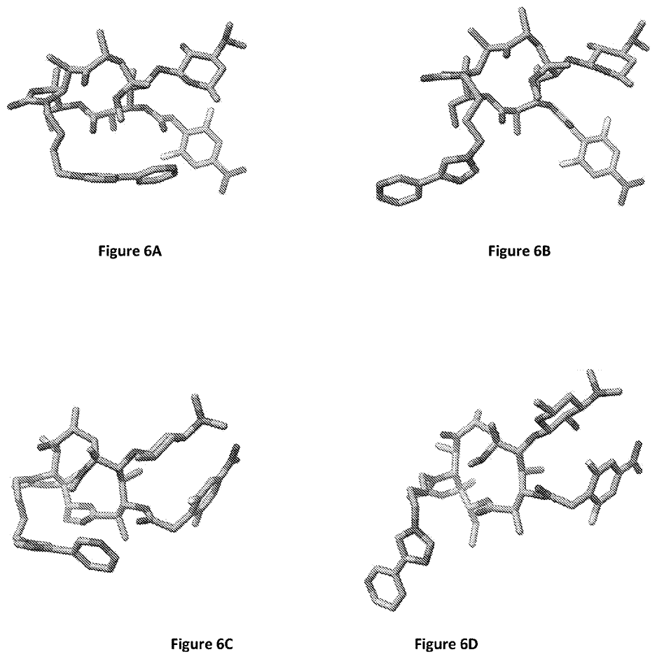

In each of the cryo-EM maps, the locations and conformations of the bound compound could be unambiguously resolved as observed from the difference electron density map (FIG. 5C-5F). The compound 56 could be modelled with four alternative conformations where the main conformation (shown in FIG. 5F), all other alternative conformations are shown in FIGS. 6A-6D.

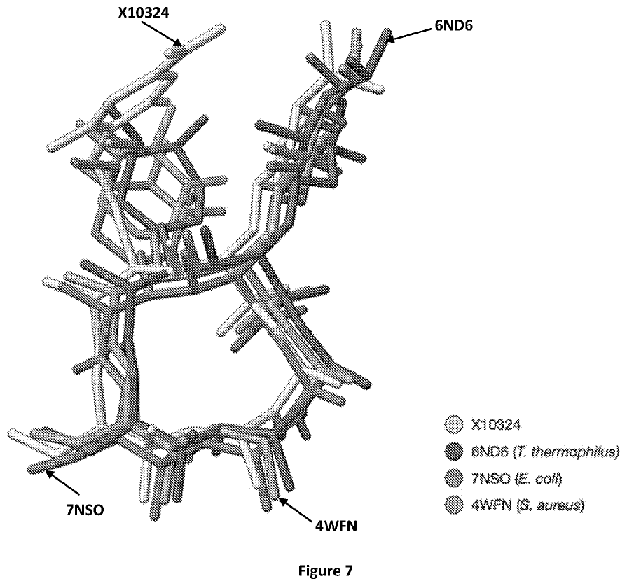

Superposition of acylides structures from each complex shows high similarity in the overall orientation of the molecules (FIG. 5B). In addition, the superposition of X10324 that was chosen as a representative acylide with ery shows a remarkable overlap (FIG. 7).

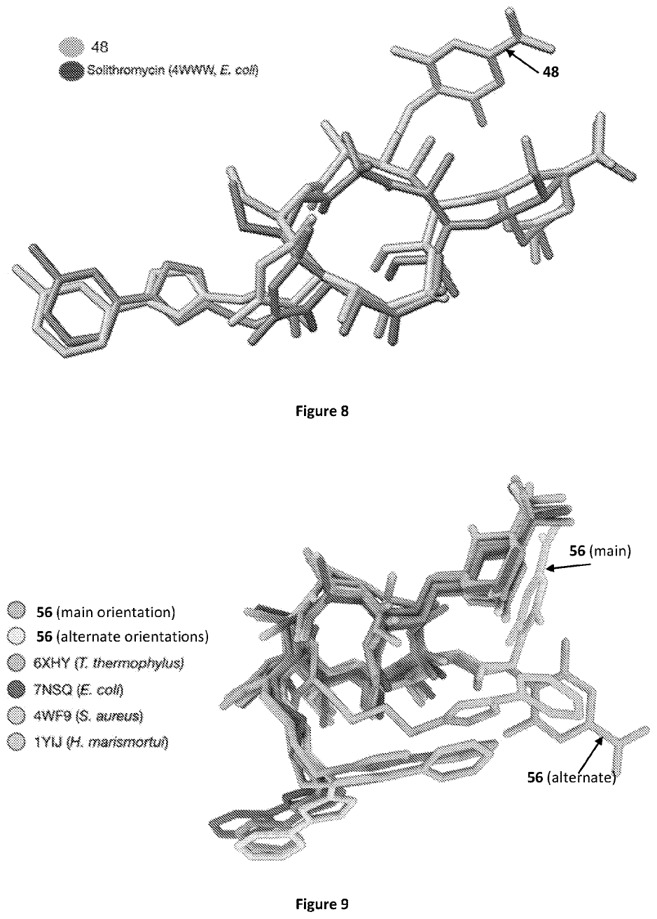

48 and 56 show a notable overlap with the existing ketolides featuring the same alkyl-aryl side chains, namely solithromycin and telithromycin (FIG. 8 and FIG. 9). Nevertheless, for 56 it could be modeled alternative orientations in addition to the classic orientation which is very similar to the other three acylides (FIGS. 5B and 8). Upon the superposition of the 56 with ribosomes in complex with telithromycin (FIG. 8) the inventors highlight the alkyl-aryl side chain, and the 2,6-difluoro-4-nitro phenylacetic acid flexibility of the 56.

### Acylides Binding Pocket Description

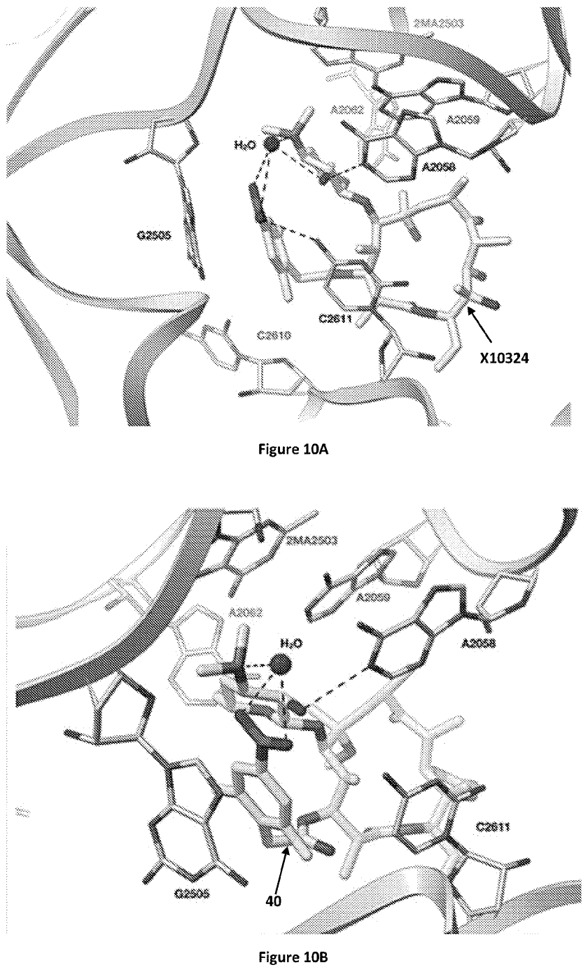

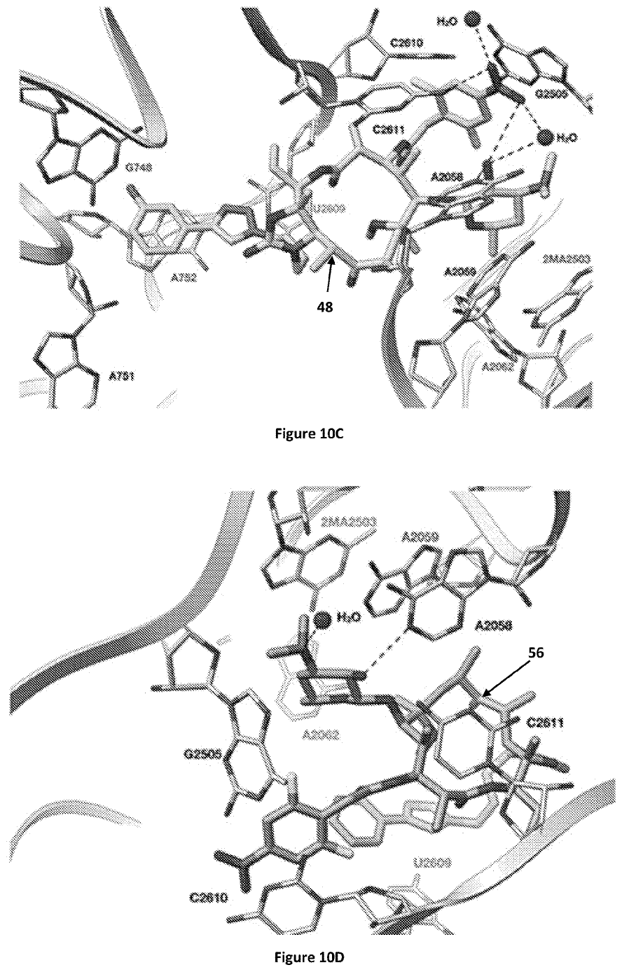

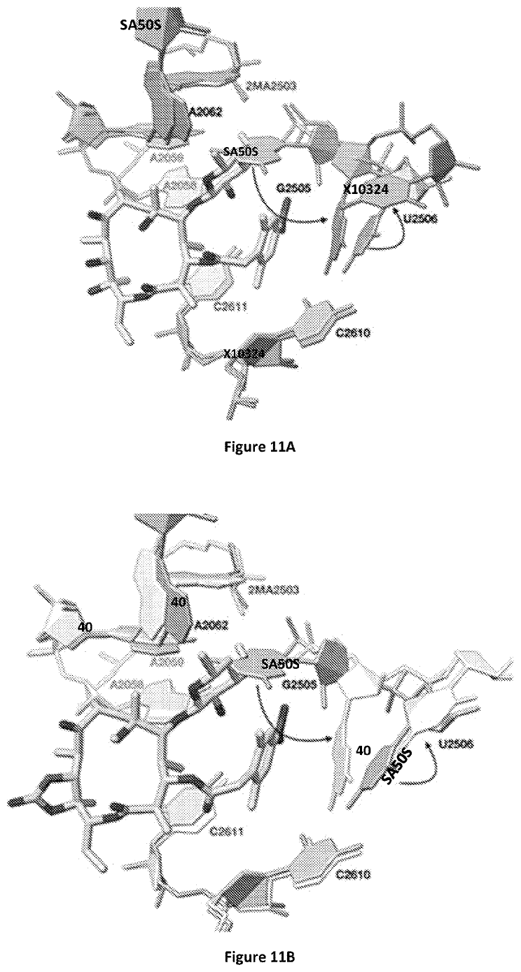

All acylides bind the ribosome at the upper part of the NPET, in a very similar orientation (FIG. 5B), and form hydrogen bonds between the desosamine C2 hydroxyl and N1 of A2058 (FIGS. 10A-10D), similarly to other macrolides.

In agreement with a previous study (Svetlov M S, Syroegin E A, Aleksandrova E V, Atkinson G C, Gregory S T, Mankin A S, Polikanov Y S. Structure of Erm-modified 70S ribosome reveals the mechanism of macrolide resistance. Nat Chem Biol. 2021 April; 17(4):412-420. doi: 10.1038/s41589-020-00715-0. Epub 2021 Jan. 18. PMLD: 33462493: PMCID: PMC7990689) a conserved water molecule appears in all of the four structures, likely mediating the hydrogen bond interactions between the N6 of A2058, the phosphate group of G2505 and the dimethylamino group of the desosamine of each acylide. The macrolactone ring and the desosamine sugar interact through Van der Waals interactions with the rRNA domain V, residues A2058, A2059, A2062, G2505, 2-methyladenosine-2503, U2609, C2610 and C2611 (FIGS. 10A-10D). Within the SA-48 complex, the orientation of the alkyl-aryl side chain forms Van der Waals interactions with rRNA domain II, helix 35, residues G748, A751, A752 (FIG. 10A), which are part of the NPET (FIG. 10C).

### Orientation of Nucleotides at the Acylides Binding Site

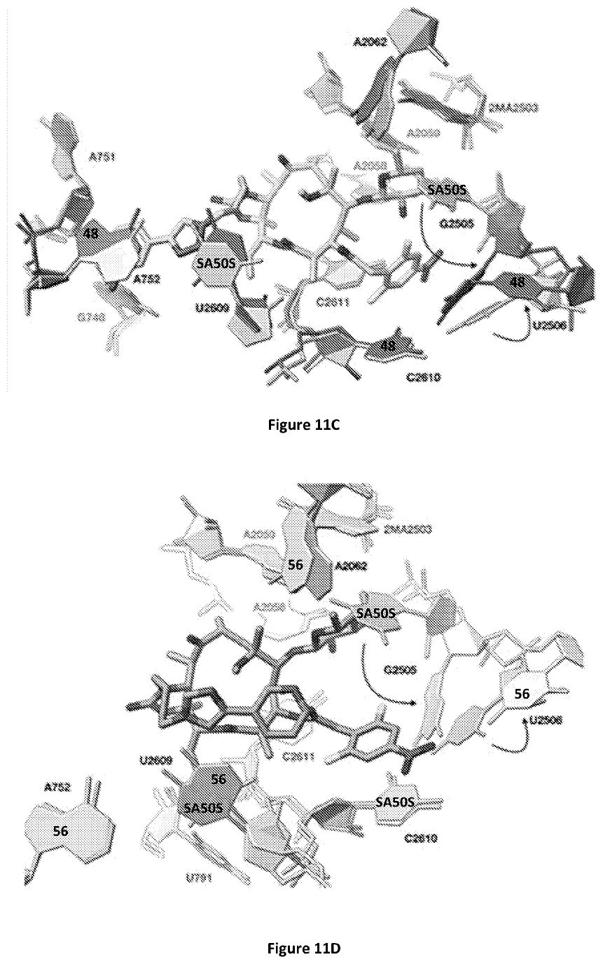

Structural changes due to acylides binding could be identified by super positioning of the complex structures of SA complexes with acylides and the apo SA ribosome structure (PDBID 6HMA) (FIG. 11A-11D). Upon acylides binding, the rRNA nucleotide G2505 is 900 rotated and followed by a rotation of the rRNA nucleotide U2506 to allow the G2502 movement. Nucleotide A2062, a known flexible moiety within the PTC (Harms, J., et al., High resolution structure of the large ribosomal subunit from a mesophilic eubacterium. Cell, 2001. 107(5): p. 679-688; Harms, J., et al., High resolution structure of the large ribosomal subunit from a mesophilic eubacterium. 2001. 107(5): p. 679-688) is flipped 90° and interacts hydrophobically with the acylides desosamine sugar. All other macrolide binding pocket nucleotides seem to be in a similar orientation to the apo SA ribosome without significant changes.

### Minimum Inhibitory Concentration Assay Using Clinical Isolates

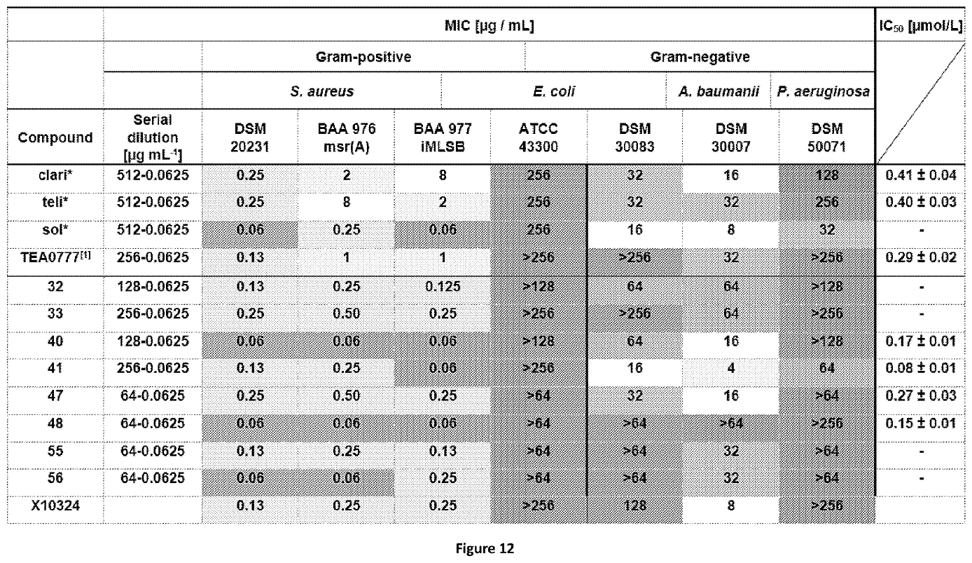

The susceptibility range of the strains in response to each compound tested is represented by a heat map (FIG. 12 (Table 8)). From these broth microdilution assays, it was concluded that acylides MIC values are superior inhibitors against gram-positive resistant SA strains and similarly to the other clinically used drugs are not active against gram-negative strains. The comparison to clarithromycin, telithromycin, solithromycin and TEA0777 highlights their clinical potential.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound represented by the structure of Formula IA: 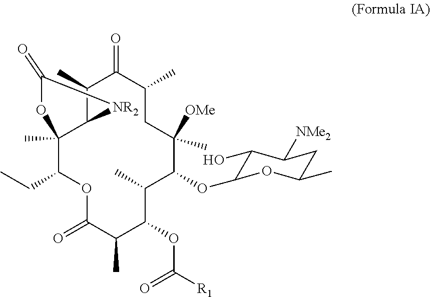
wherein
R₁ is 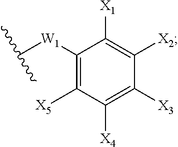
X₁-X₅ are each independently selected from H, halo, CN, NO₂, OH, —O-alkylene-aryl, —O— alkylene-heteroaryl, —N-alkylene-aryl, —N-alkylene-heteroaryl, —SO₂-alkylene-aryl, and —SO₂— alkylene-heteroaryl; or two of X₁-X₅ are connected via —O—(CH₂)ₘ—O—; wherein at least one of X₁-X₅ is not H;
m is an integer from 1-10;
W₁ is a alkylene;
R₂ is H or 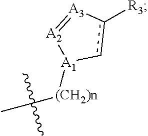
wherein if R₂ is H, R₁ is not p —CH₂—C₆H₄—NO₂; [symbol]
represents a single or double bond;
n is an integer from 0-10;
R₃ is heteroaryl or aryl, wherein R₃ is not pyrimidine; wherein if X₃ is Cl, R₃ is not phenyl;
A₁, A₂, and A₃ are each independently N, NH, NR′, C, CH, CR″, CHR″, CR″R′″ or CH₂; and
R′, R″, and R′″ are each independently alkyl, cycloalkyl, aryl, heteroaryl, NO₂, CN, hydroxyl or halo.

2. The compound of claim 1, wherein the compound is represented by the structure of Formula IB:

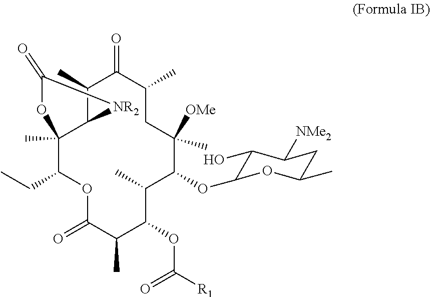

3. The compound of claim 2, wherein the compound represented by the structure of Formula IC:

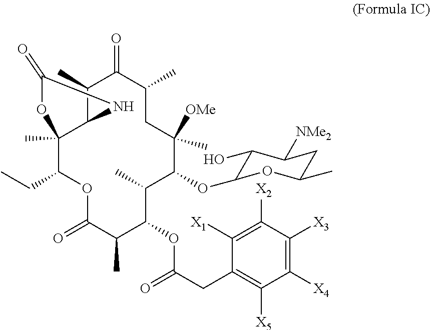

4. The compound of claim 2, wherein the compound is represented by the structure of Compounds-29-33 and-35-42:

| Compound's | |
|---|---|
| Number | R₁ of Formula IB |
| 29 | 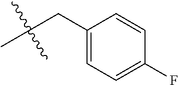 [SMILES: *Cc1ccc(F)cc1] |
| 30 | 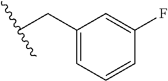 [SMILES: *Cc1cccc(F)c1] |
| 31 | 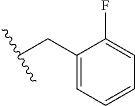 [SMILES: *Cc1ccccc1F] |
| 32 | 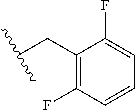 [SMILES: *Cc1c(F)cccc1F] |
| 33 | 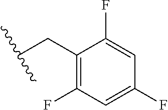 [SMILES: *Cc1c(F)cc(F)cc1F] |
| 35 | 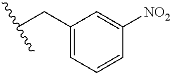 [SMILES: *Cc1cccc([N+](=O)[O-])c1] |
| 36 | 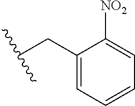 [SMILES: *Cc1ccccc1[N+](=O)[O-]] |
| 37 | 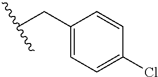 [SMILES: *Cc1ccc(Cl)cc1] |
| 38 | 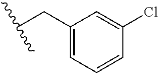 [SMILES: *Cc1cccc(Cl)c1] |
| 39 | 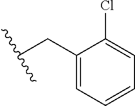 [SMILES: *Cc1ccccc1Cl] |
| 40 | 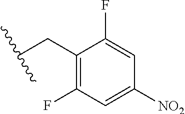 [SMILES: *Cc1c(F)cc([N+](=O)[O-])cc1F] |
| 41 | 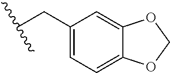 [SMILES: *Cc1ccc2c(c1)OCO2] |
| 42 | 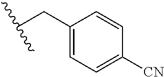 [SMILES: *Cc1ccc(C#N)cc1] |

5. The compound of claim 1, wherein the compound is represented by the structure of Formula ID:

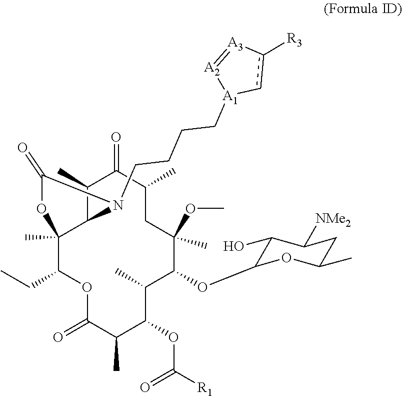

6. The compound of claim 5, wherein the compound is represented by the structure of Formula IE:

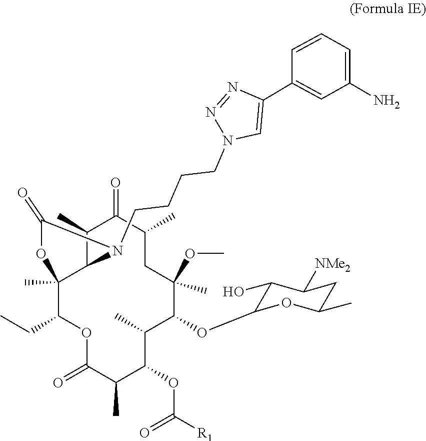

7. The compound of claim 6, wherein the structure is represented by Compounds 47-48:

| Compound's | |
|---|---|
| Number | R₁ of Formula IE |
| 47 | 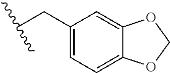 [SMILES: *Cc1ccc2c(c1)OCO2] |
| 48 | 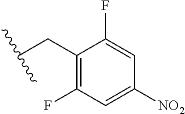 [SMILES: *Cc1c(F)cc([N+](=O)[O-])cc1F] |

8. The compound of claim 5, wherein the compound is represented by the structure of Formula IF:

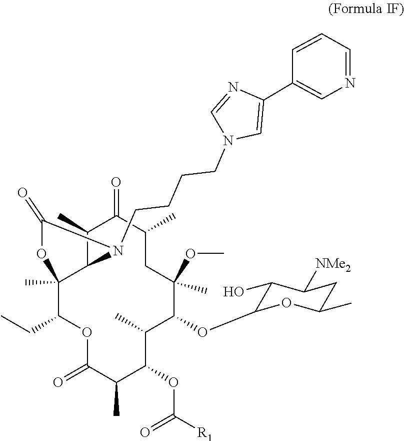

9. The compound of claim 8, wherein the structure is represented by Compounds 55-56:

| Compound's | |
|---|---|
| Number | R₁ of Formula IF |
| 55 | 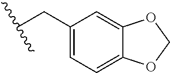 [SMILES: *Cc1ccc2c(c1)OCO2] |

10. A compound represented by the structure of Formula IG: 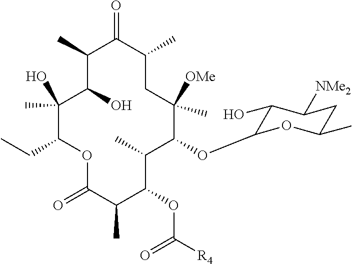
wherein
R₄ is 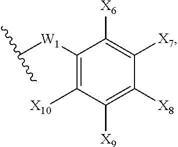
-alkylene-N₃, -alkylene-heterocycloalkyl-aryl, -alkylene-heterocycloalkyl, -alkylene-heterocycloalkyl-aryl, -alkylene-heteroaryl-aryl, -alkylene-heteroaryl-alkylene-heterocycloalkyl, -alkylene-heteroaryl-alkylene-aryl;
X₆-X₁₀ are each independently selected from H, F, I, Br, CN, OH, O— alkylene-aryl, —O— alkylene-heteroaryl, —SO₂-alkylene-aryl, —SO₂-alkylene-heteroaryl;
wherein at least one of X₆-X₁₀ is not hydrogen;
wherein if X₆ and X₁₀ are F, then at least one of X₇-X₉ is not H or F; and
W₁ is an alkylene.

11. The compound of claim 10, wherein the structure is represented by Compounds 2-5, 7-13, 16, and 18-24:

| Compound's | |
|---|---|
| Number | R4 of Formula IG |
| 2 | 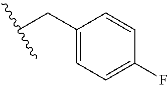 [SMILES: *Cc1ccc(F)cc1] |
| 3 | 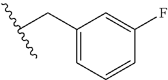 [SMILES: *Cc1cccc(F)c1] |
| 4 | 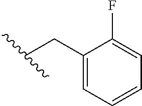 [SMILES: *Cc1ccccc1F] |
| 5 | 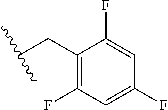 [SMILES: *Cc1c(F)cc(F)cc1F] |
| 7 | 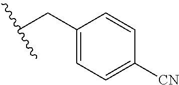 [SMILES: *Cc1ccc(C#N)cc1] |
| 8 | 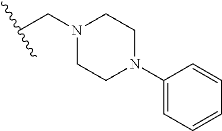 [SMILES: *CN1CCN(c2ccccc2)CC1] |
| 9 | 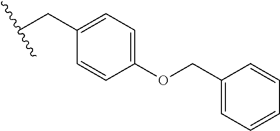 [SMILES: *Cc1ccc(OCc2ccccc2)cc1] |
| 10 | 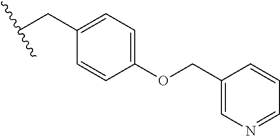 [SMILES: *Cc1ccc(OCc2cccnc2)cc1] |
| 11 | 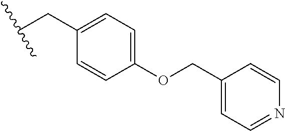 [SMILES: *Cc1ccc(OCc2ccncc2)cc1] |
| 12 | 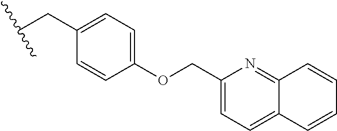 [SMILES: *Cc1ccc(OCc2ccc3ccccc3n2)cc1] |
| 13 | 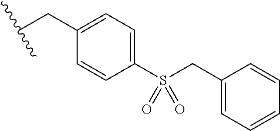 [SMILES: *Cc1ccc(S(=O)(=O)Cc2ccccc2)cc1] |
| 16 | 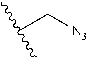 [SMILES: *CN=[N+]=[N-]] |
| 18 | 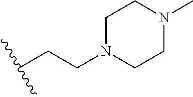 [SMILES: *CCN1CCN(C)CC1] |
| 19 | 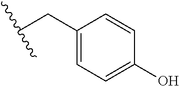 [SMILES: *Cc1ccc(O)cc1] |
| 20 | 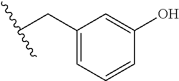 [SMILES: *Cc1cccc(O)c1] |
| 21 | 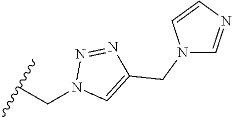 [SMILES: *Cn1cc(Cn2ccnc2)nn1] |
| 22 | 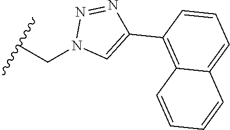 [SMILES: *Cn1cc(-c2cccc3ccccc23)nn1] |
| 23 | 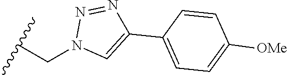 [SMILES: *Cn1cc(-c2ccc(OC)cc2)nn1] |
| 24 | 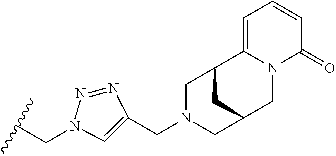 [SMILES: *Cn1cc(CN2C[C@@H]3C[C@H](C2)c2cccc(=O)n2C3)nn1] |

12. A pharmaceutical composition comprising a compound according to claim 1, and a pharmaceutically acceptable carrier.

13. A method for inhibiting selectively S. aureus resistant strains comprising administering the pharmaceutical composition of claim 12, wherein the strain is DSM20231, BAA976, or BAA977.

14. The method of claim 13 wherein the compound is any one of compounds 29-42, 47-48, and 55-56.

15. A method for inhibiting selectively of E. coli strain, comprising administering the pharmaceutical composition of claim 12, wherein the strain is tolC; MB5747.

16. The method of claim 15, wherein the compound is any one of compounds 29-42, 47-48, and 55-56.

17. A method for the treatment of an infection comprising administering the pharmaceutical composition of claim 12, wherein the infection is due to E. coli strain, and wherein the strain is tolC; MB5747; or the infection is due to S. aureus resistant strains, wherein the strain is DSM20231, BAA976 or BAA977.

18. A pharmaceutical composition comprising a compound according to claim 10, and a pharmaceutically acceptable carrier.

19. A method for inhibiting selectively S. aureus resistant strains comprising administering the pharmaceutical composition of claim 18, wherein the strain is DSM20231, BAA976, or BAA977.

20. A method for inhibiting selectively E. coli strain comprising administering the pharmaceutical composition of claim 18, wherein the strain is tolC; MB5747.

21. A method for the treatment of an infection comprising administering the pharmaceutical composition of claim 18, wherein the infection is due to S. aureus resistant strains, wherein the strain is DSM20231, BAA976 or BAA977; or the infection is due to E. coli strain, wherein the strain is tolC: MB5747.

22-30. (canceled)
